(19) United States
(12) Patent Application Publication
(10) Pub. No.: US 2025/0270577 A1
(43) Pub. Date: Aug. 28, 2025

(54) METHODS OF MODULATING SEED AND ORGAN SIZE IN PLANTS

(71) Applicant: Plant Bioscience Limited, Norwich (GB)

(72) Inventors: MICHAEL BEVAN, NORWICH (GB); JACK DUMENIL, NORWICH (GB)

(21) Appl. No.: 19/038,251

(22) Filed: Jan. 27, 2025

Related U.S. Application Data
(62) Division of application No. 17/305,967, filed on Jul. 19, 2021, now Pat. No. 12,247,210.
Application No. 17/305,967 is a continuation of application No. 16/405,740, filed on May 7, 2019, now Pat. No. 11,118,188.
Application No. 16/405,740 is a continuation of application No. 15/035,657, filed on May 10, 2016, now Pat. No. 10,337,021.
Application No. 15/035,657 is a continuation of application No. PCT/GB14/53296, filed on Nov. 5, 2014.

(30) Foreign Application Priority Data
Nov. 11, 2013 (GB) 1319876.7

(51) Int. Cl. C12N 15/82; A01H 5/00; A01H 5/08; A01H 5/10; C07K 14/415

(52) U.S. Cl. CPC C12N 15/8261; A01H 5/00; A01H 5/08; A01H 5/10; C07K 14/415; Y02A 40/146

(57) ABSTRACT

This invention relates to the expression a DA1 protein with a mutation that disrupts or inactivates the LIM domain or the LIM-like domain within cells of a plant. This may increase the yield or enhance a yield-related trait of the plant. Methods, plants and plant cells are provide.

### CROSS REFERENCE TO RELATED APPLICATIONS

This is a divisional application of U.S. Ser. No. 17/305,967, filed Jul. 19, 2021, which is a Continuation Application of U.S. Ser. No. 16/405,740, filed May 7, 2019, now U.S. Pat. No. 11,118,188, issued Sep. 14, 2021, which is a Continuation Application of U.S. Ser. No. 15/035,657, filed May 10, 2016, now U.S. Pat. No. 10,337,021, issued Jul. 2, 2019, which claims priority to PCT/GB2014/053296, filed Nov. 5, 2014, and GB 1319876.7, filed Nov. 11, 2013, all of which are herein incorporated by reference in their entireties.

### SEQUENCE LISTING

The instant application contains a Sequence Listing which has been submitted in ASCII format via EFS-Web and is hereby incorporated by reference in its entirety. Said ASCII copy, created on Jul. 16, 2019, is named BEVAN_P11855US01_SEQ_LISTING_07-16-19_ST25.txt and is 174,398 bytes in size.

### FIELD OF INVENTION

This invention relates to methods of altering the size of the seeds and organs of plants, for example to improve plant yield.

### BACKGROUND OF INVENTION

The size of seeds and organs is an agronomically and ecologically important trait that is under genetic control (Alonso-Blanco, C. PNAS USA 96, 4710-7 (1999); Song, X.J. Nat Genet 39, 623-30 (2007); Weiss, J. Int J Dev Biol 49, 513-25 (2005); Dinneny, J.R. Development 131, 1101-10 (2004); Disch, S. Curr Biol 16, 272-9 (2006); Science 289, 85-8 (2000); Horiguchi, G. Plant J 43, 68-78 (2005); Hu, Y Plant J 47, 1-9 (2006); Hu, Y. Plant Cell 15, 1951-61 (2003); Krizek, B.A. Dev Genet 25, 224-36 (1999); Mizukami, Y. PNAS USA 97, 942-7 (2000); Nath, U. Science 299, 1404-7 (2003); Ohno, C.K. Development 131, 1111-22 (2004); Szecsi, J. Embo J 25, 3912-20 (2006); White, D.W. PNAS USA 103, 13238-43 (2006); Horvath, B.M. Embo J 25, 4909-20 (2006); Garcia, D. Plant Cell 17, 52-60 (2005). The final size of seeds and organs is constant within a given species, whereas interspecies seed and organ size variation is remarkably large, suggesting that plants have regulatory mechanisms that control seed and organ growth in a coordinated and timely manner. Despite the importance of seed and organ size, however, little is known about the molecular and genetic mechanisms that control final organ and seed size in plants.

The genetic regulation of seed size has been investigated in plants, including in tomato, soybean, maize, and rice, using quantitative trait locus (QTL) mapping. To date, in the published literature, two genes (Song, X.J. Nat Genet 39, 623-30 (2007); Fan, C. Theor. Appl. Genet. 112, 1164-1171 (2006)), underlying two major QTLs for rice grain size, have been identified, although the molecular mechanisms of these genes remain to be elucidated. In Arabidopsis, eleven loci affecting seed weight and/or length in crosses between the accessions Ler and Cvi, have been mapped {Alonso-Blanco, 1999 supra}, but the corresponding genes have not been identified. Recent studies have revealed that AP2and ARF2 are involved in control of seed size. Unfortunately, however, ap2 and arf2 mutants have lower fertility than wild type (Schruff, M.C. Development 137, 251-261 (2006); Ohto, M.A. PNAS USA 102, 3123-3128 (2005); Jofuku, K.D. PNAS USA 102, 3117-3122 (2005)). In addition, studies using mutant plants have identified several positive and negative regulators that influence organ size by acting on cell proliferation or expansion {Krizek, B.A. Dev Genet 25, 224-36 (1999); Mizukami, Y. Proc Natl Acad Sci USA 97, 942-7 (2000); Nath, U. Science 299, 1404-7 (2003); Ohno, C.K. Development 131, 1111-22 (2004); Szecsi, J. Embo J 25, 3912-20 (2006); White, D.W. PNAS USA 103, 13238-43 (2006); Horvath, B.M. Embo J 25, 4909-20 (2006); Garcia, D. Plant Cell 17, 52-60 (2005). Horiguchi, G. Plant J 43, 68-78 (2005); Hu, Y Plant J 47, 1-9 (2006) Dinneny, J.R. Development 131, 1101-10 (2004)).

Several factors involved in ubiquitin-related activities have been known to influence seed size. A growth-restricting factor, DA1, is a ubiquitin receptor and contains two ubiquitin interaction motifs (UIMs) that bind ubiquitin in vitro, and da1-1 mutant forms large seeds by influencing the maternal integuments of ovules (Li et al., 2008). Mutations in an enhancer of da1-1 (EOD1), which encodes the E3 ubiquitin ligase BIG BROTHER (BB) (Disch et al., 2006; Li et al., 2008), synergistically enhance the seed size phenotype of da1-1, indicating that DA1 acts synergistically with EOD1/BB to control seed size.

Identification of further factors that control the final size of both seeds and organs will not only advance understanding of the mechanisms of size control in plants, but may also have substantial practical applications for example in improving crop yield and plant biomass for generating biofuel.

### SUMMARY OF INVENTION

The present inventors have unexpectedly discovered that disruption of the LIM domain and/or the LIM-like domain in plant DA1 proteins does not abolish DA homodimerisation or activity but instead confers a dominant-negative phenotype.

An aspect of the invention provides a method of increasing the yield of a plant or enhancing a yield-related trait in a plant; comprising expressing a DA1 protein having an inactivated LIM domain or LIM-like domain within cells of said plant.

The DA1 protein may comprise one or more mutations relative to the wild-type sequence that disrupt or inactivate the LIM domain or LIM-like domain of the DA1 protein.

Expression of a DA1 protein with a disrupted or inactivated LIM domain or LIM-like domain enhances one or more yield related traits and increases the yield of the plant.

The DA1 protein having an inactivated LIM domain or LIM-like domain may be expressed from a heterologous nucleic acid coding sequence in one or more cells of the plant or may be expressed from an endogenous nucleic acid coding sequence in one or more cells of the plant.

Another aspect of the invention provides a method of producing a plant with an increased yield and/or one or more enhanced yield-related traits comprising:
introducing into a plant cell a heterologous nucleic acid which encodes a DA1 protein having an inactivated LIM domain or LIM-like domain, or
introducing a mutation into the nucleotide sequence of a plant cell which encodes the DA1 protein, such that the LIM domain or LIM-like domain of the DA1 protein is inactivated, and
regenerating the plant from the plant cell.

Another aspect of the invention provides a plant cell comprising a heterologous nucleic acid encoding a DA1 protein having an inactivated LIM domain or LIM-like domain.

Another aspect of the invention provides a plant comprising one or more plant cells that comprise a heterologous nucleic acid encoding a DA1 protein having an inactivated LIM domain or LIM-like domain.

The plant may display increased yield or an enhanced a yield-related trait relative to controls.

### BRIEF DESCRIPTION OF DRAWINGS

FIG. 1 shows the spacing and identity of the eight zinc binding residues (1-8) in the LIM domain based on an analysis of 135 human LIM sequences. Infrequently observed patterns (<10%) of conserved sequence and topography of the LIM domain.

FIG. 2 shows the topology of Zn coordination of a LIM domain. Numbered circles indicate Zn binding residues. Semi conserved aliphatic/bulky residues are shown with horizontal lines, non-conserved residues with invariant spacing are shown with vertical lines. Circles with a dashed border indicate a variable number of residues (X) that are possible within the space.

FIG. 3 shows in vitro immunoprecipitation that shows the binding of da1lim8 to wild-type DA1. E. coli expressed GST-tagged bait proteins were incubated with E. coli expressed FLAG-tagged prey proteins before purification on glutathione sepharose beads and immunoblotting for GST and FLAG. FLAG-DA1 and FLAG-da1lim8 co-purified with GST-DA1 and GST-da1lim8 (lanes 5,6,8,9) but not with the negative control GST-GUS (lanes 2,3); revealing that mutating the LIM domain in DA1 is not sufficient to abolish the physical interaction between DA1 proteins.

FIG. 4 shows the effect of the lim8 mutation on seed size in a Columbia background.

FIGS. 5A-O show an alignment of DA1 proteins (SEQ ID NOS: 4 to 27).

FIGS. 6A-C shows an alignment of EOD1 proteins (SEQ ID NOS: 38 to 53).

### DETAILED DESCRIPTION OF EMBODIMENTS OF THE INVENTION

This invention relates the expression in plants of DA1 proteins in which the LIM or LIM-like domain is disrupted or inactivated (collectively termed LIM-disrupted DA1 proteins herein). This may be useful in altering plant traits which affect yield, such as seed and organ size.

DA1 is a plant ubiquitin receptor that is described in detail in Li et al (2008), Wang, et al (2012) and WO2009/047525.

DA1 proteins are characterised by the presence of a LIM domain, a LIM-like domain, a conserved C terminal domain and one or more UIM domains.

A LIM domain comprises two Zn finger motifs and may have the amino acid sequence (SEQ ID NO:1);

C(X)₂C(X)₁₆₋₂₃(H/C)(X)_{2/4}(C/H/E)(X)₂C(X)₂
C(X)₁₄₋₂₁(C/H)(X)_{2/1/3}(C/H/D/E)X

where X is any amino acid and Zn coordinating residues are underlined.

The Zn coordinating residues in the LIM domain may be C, H, D or E, preferably C.

In some preferred embodiments, a LIM domain may comprise CXXC, HXXCXXCXXC and HxxC motifs, where X is any amino acid. For example, a LIM domain may comprise the amino acid sequence (SEQ ID NO:2);

C(X)₂C(X)₁₆₋₂₃(H)(X)₂(C)(X)₂C(X)₂C(X)₁₄₋₂₁H(X)₂CX

where X is any amino acid and Zn coordinating residues are underlined.

In some embodiments, a LIM domain may comprise the amino acid sequence of the AtDA1 LIM domain;

CAGCNMEIGHGRFLNCLNSLWHPECFRCYGCSQPISEYEFSTS
GNYPFHKACY

(SEQ ID NO: 3; Zn coordinating residues are underlined)

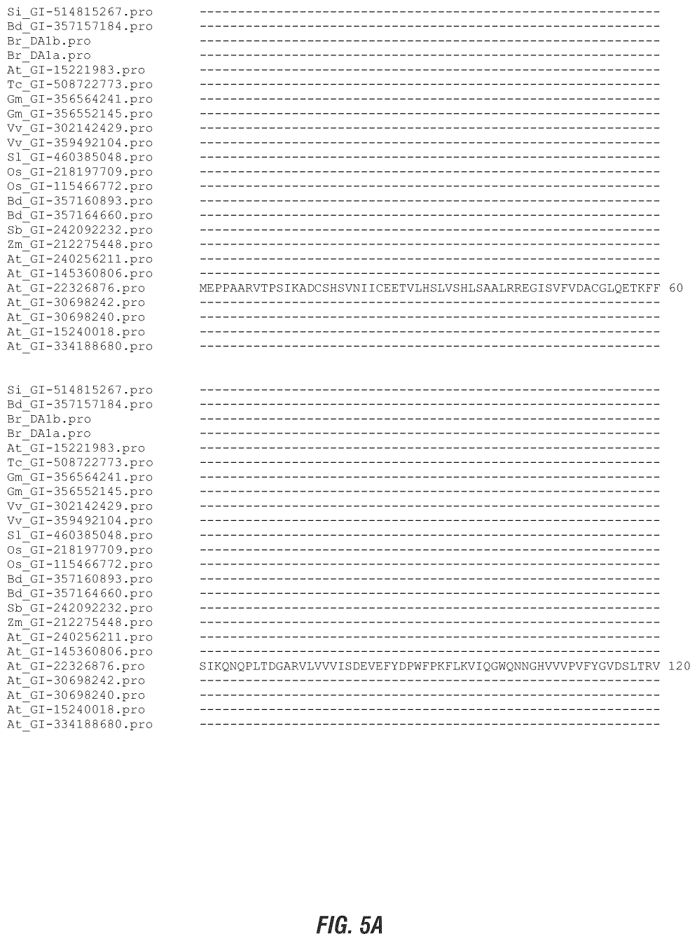

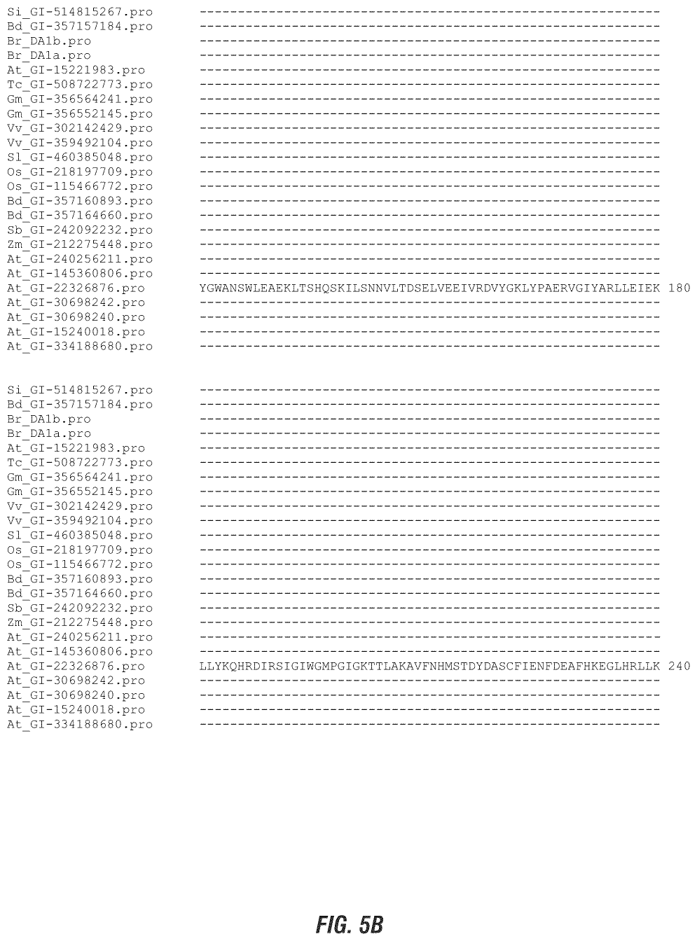

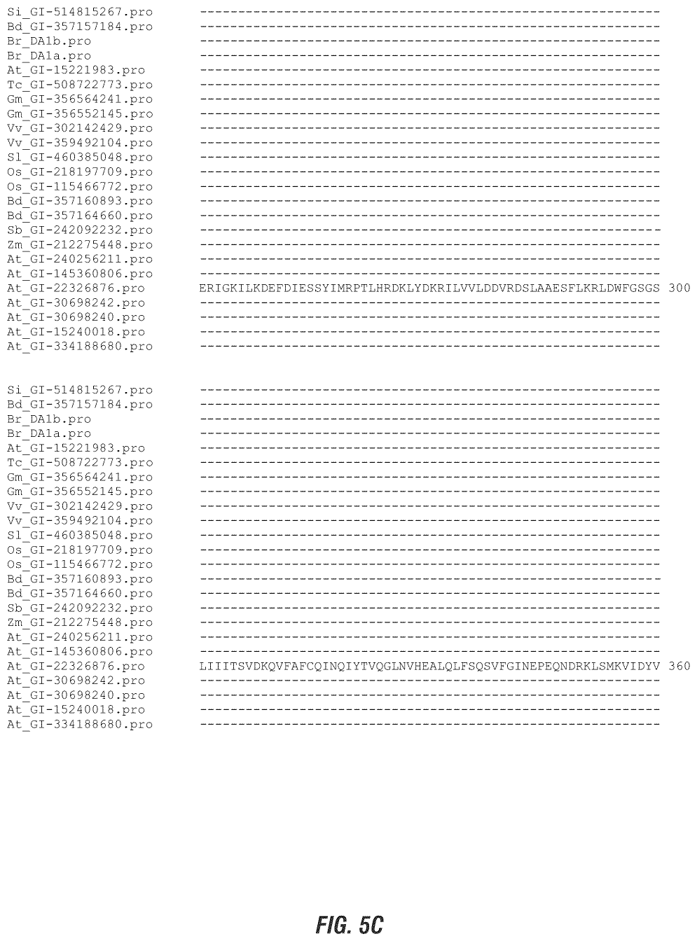

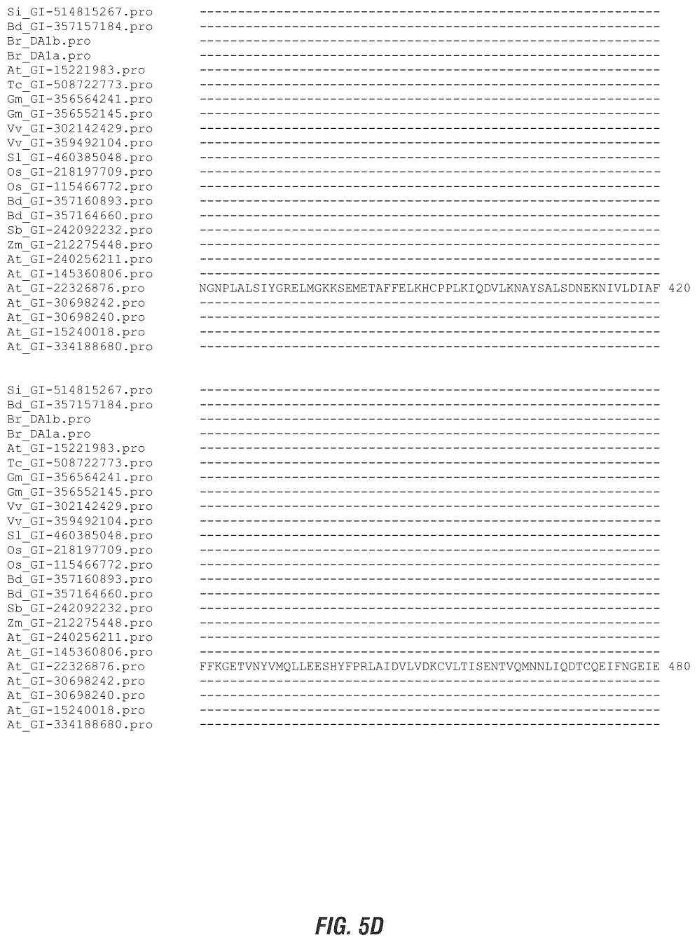

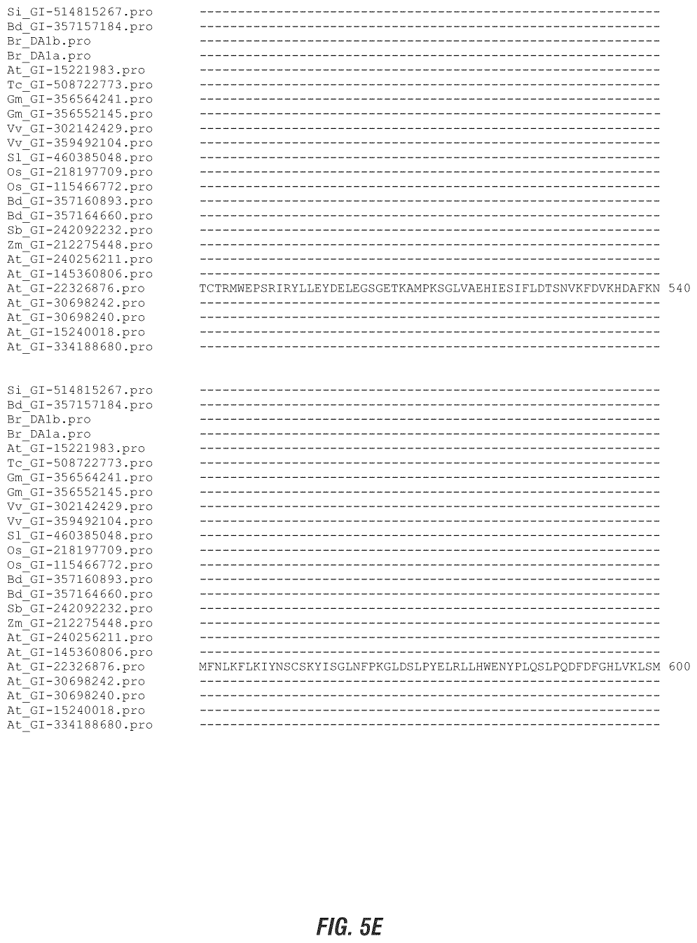

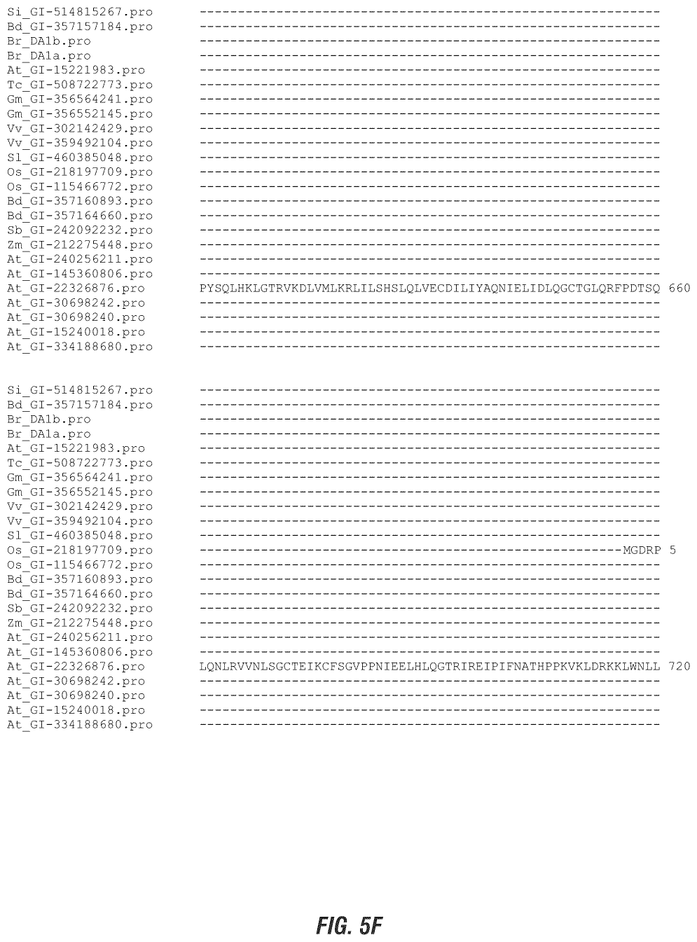

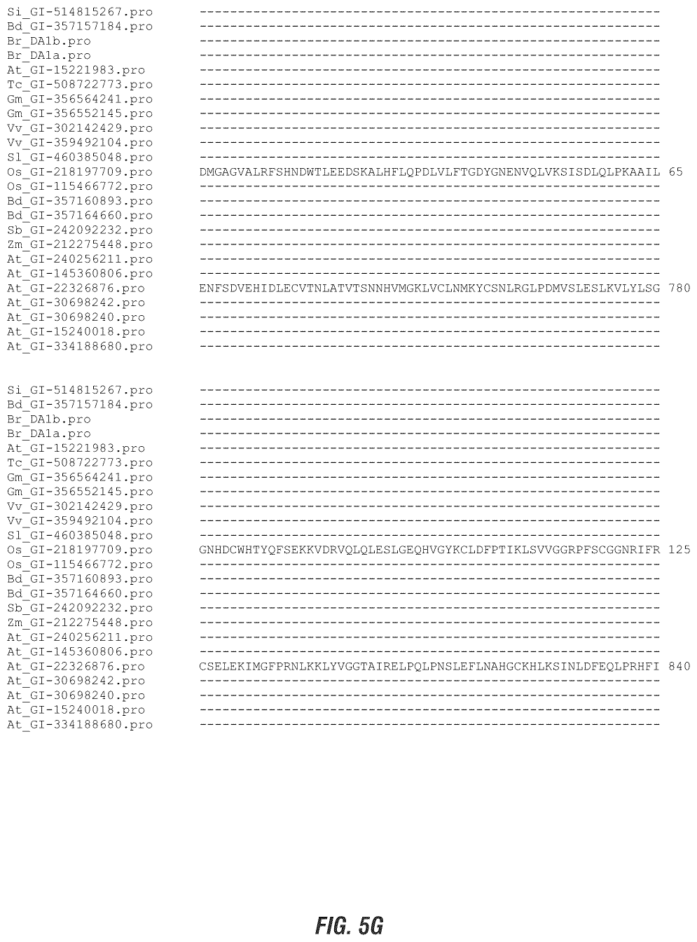

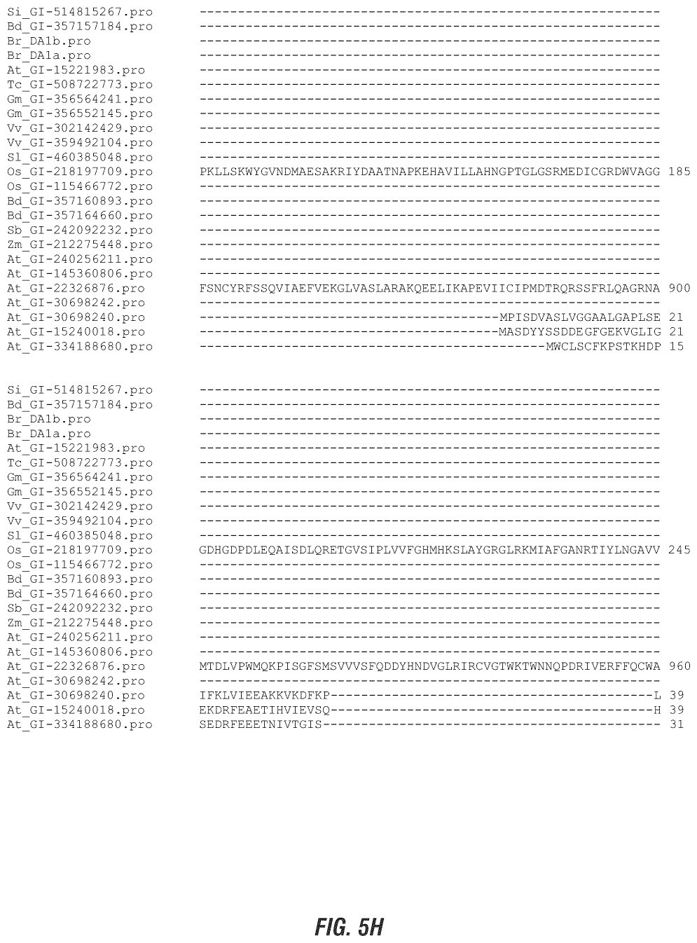

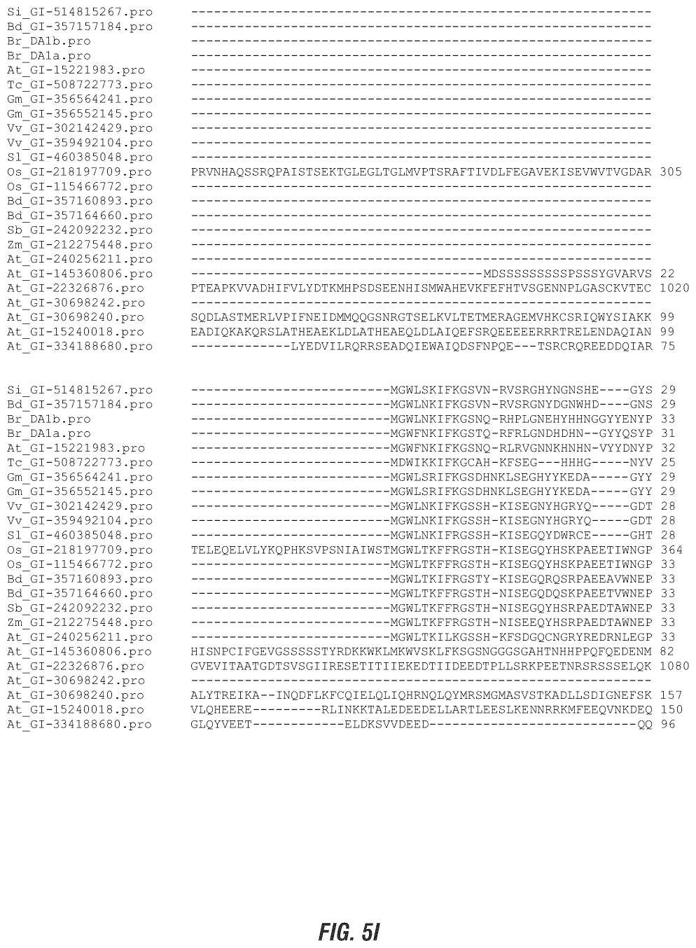

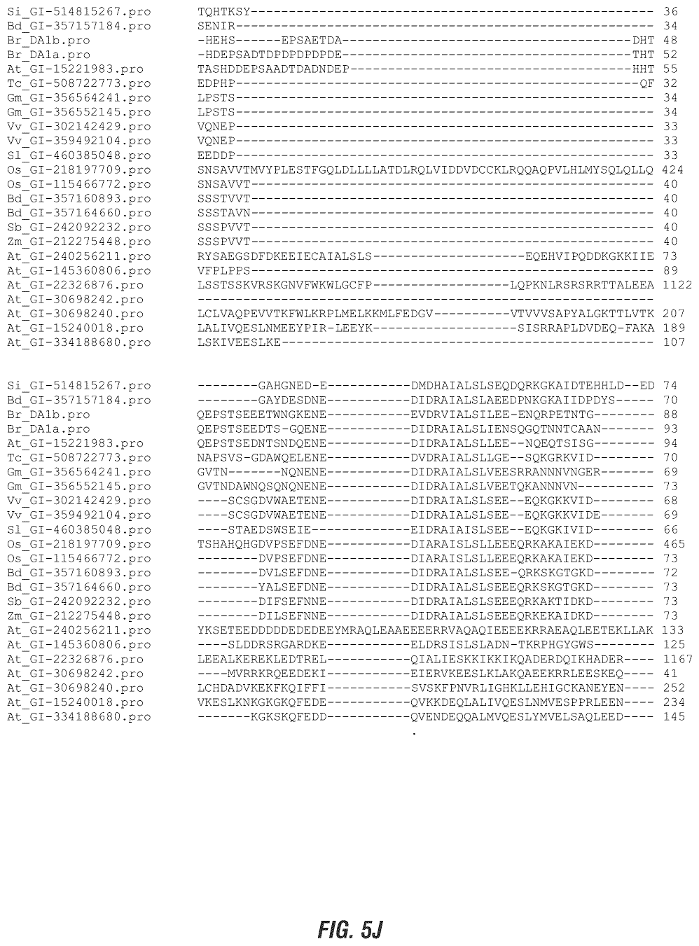

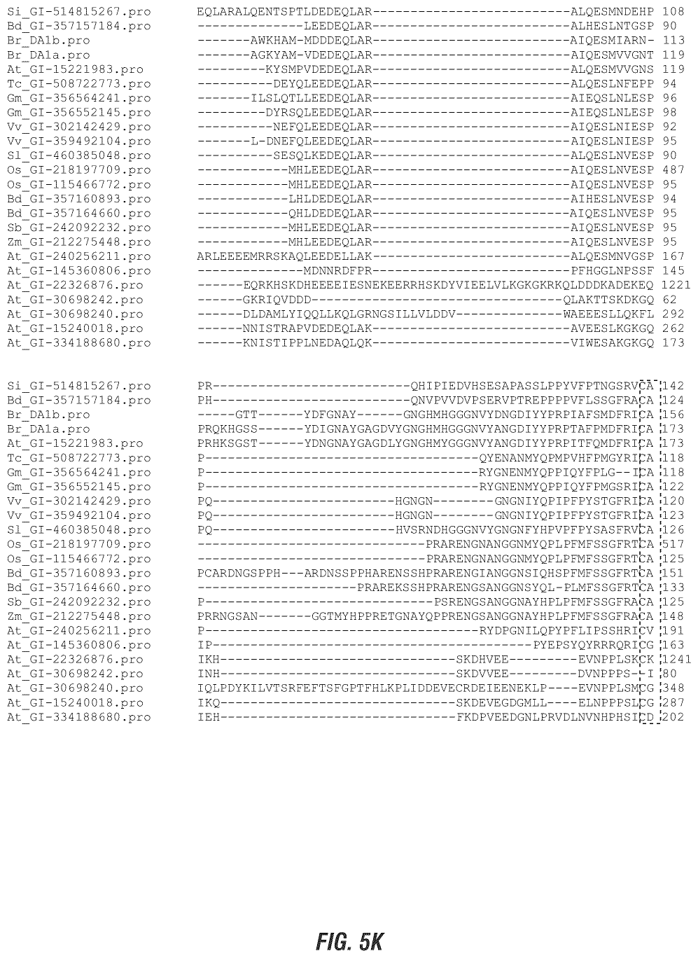

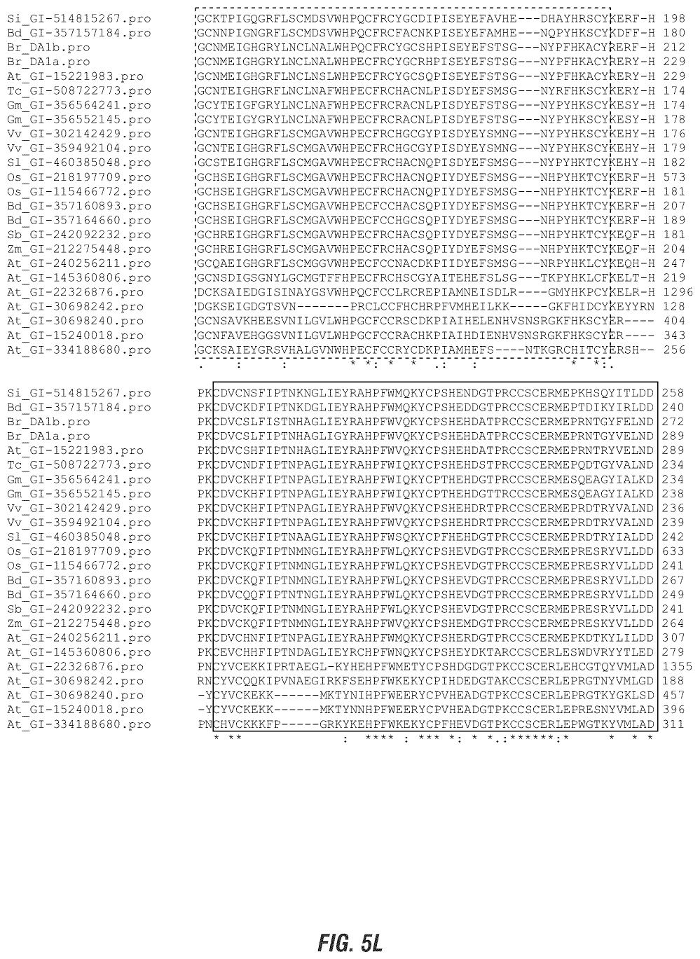

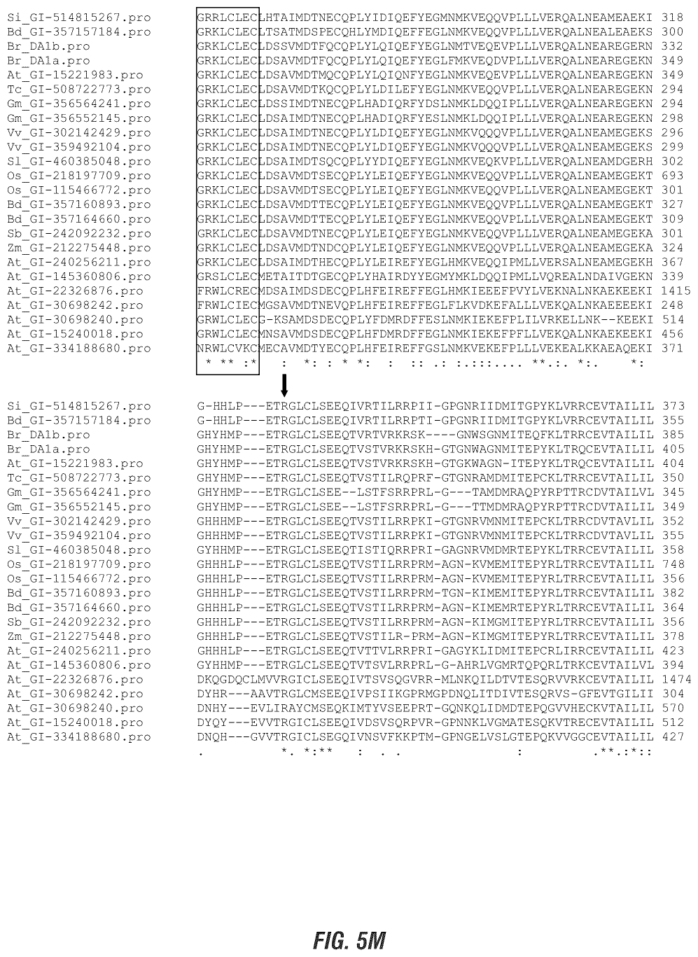

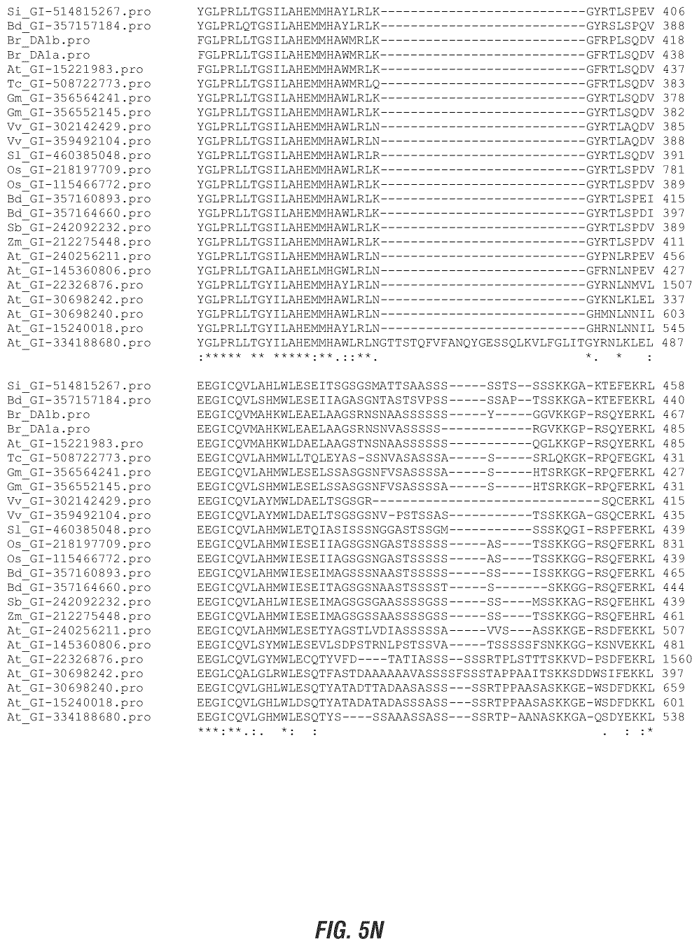

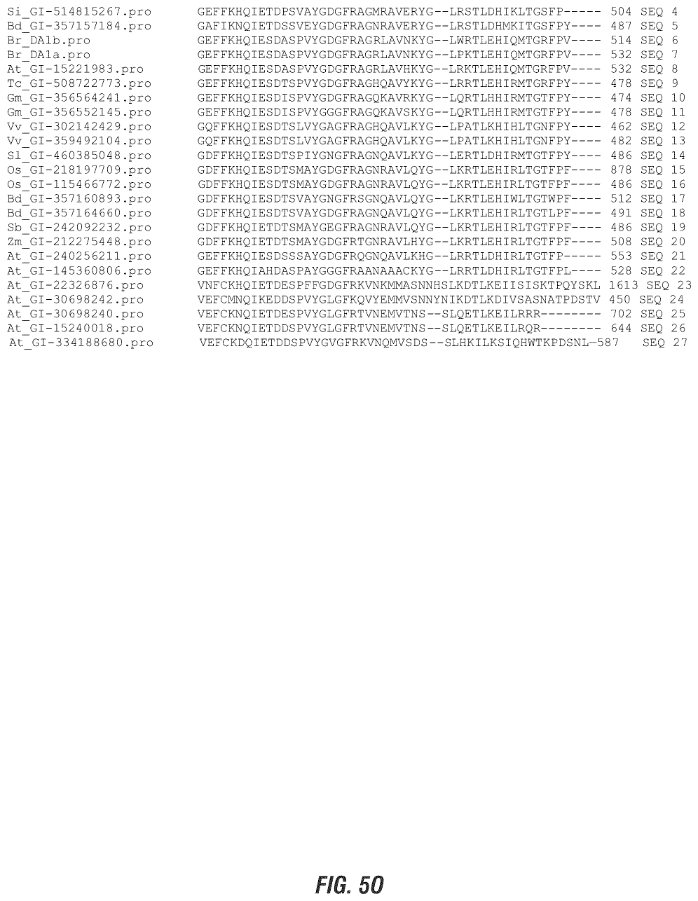

Other LIM domains include the LIM domain of an DA1 amino acid sequence shown in FIG. 5 (dashed box), for example residues 141 to 193 of SEQ ID NO: 4 (Si_GI-514815267.pro), residues 123 to 175 of SEQ ID NO: 5 (Bd_GI-357157184.pro), residues 155 to 207 of SEQ ID NO: 6(Br_DA1b.pro), residues 172 to 224 of SEQ ID NO: 7 (Br_DA1a.pro), residues 172 to 224 of SEQ ID NO: 8 (At_GI-15221983.pro), residues 117 to 169 of SEQ ID NO: 9 (Tc_GI-508722773.pro), residues 117 to 169 of SEQ ID NO: 10 (Gm_GI-356564241.pro), residues 121 to 173 of SEQ ID NO: 11 (Gm_GI-356552145.pro), residues 119 to 171 of SEQ ID NO: 12 (Vv_GI-302142429.pro), residues 122 to 174 of SEQ ID NO: 13 (Vv_GI-359492104.pro), residues 125 to 177 of SEQ ID NO: 14 (SI_GI-460385048.pro), residues 516 to 568 of SEQ ID NO: 15 (Os_GI-218197709.pro), residues 124 to 176 of SEQ ID NO: 16 (Os_GI-115466772.pro), residues 150 to 202 of SEQ ID NO: 17 (Bd_GI-357160893.pro), residues 132 to 184 of SEQ ID NO: 18 (Bd_GI-357164660.pro), residues 124 to 176 of SEQ ID NO: 19 (Sb_GI-242092232.pro), residues 147 to 199 of SEQ ID NO: 20 (Zm_GI-212275448.pro), residues 190 to 242 of SEQ ID NO: 21 (At_GI-240256211.pro), residues 162 to 214 of SEQ ID NO: 22 (At_GI-145360806.pro), residues 1240 to 1291 of SEQ ID NO: 23 (At_GI-22326876.pro), residues 80 to 122 of SEQ ID NO: 24 (At_GI-30698242.pro), residues 347 to 402 of SEQ ID NO: 25 (At_GI-30698240.pro), residues 286 to 341 of SEQ ID NO: 26 (At_GI-15240018.pro) or residues 202 to 252 of SEQ ID NO: 27 (At_GI-334188680.pro).

LIM domain sequences may be identified using standard sequence analysis techniques (e.g. Simple Modular Architecture Research Tool (SMART); EMBL Heidelberg, DE).

A LIM-like domain comprises two Zn finger motifs and may comprise CXXC, HXXXXXXXCXXH and CxxC motifs, where X is any amino acid. For example, a LIM-like domain may comprise the amino acid sequence (SEQ ID NO:28);

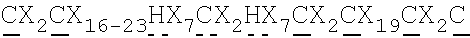

where X is any amino acid, Zn coordinating residues are solid underlined and putative Zn coordinating residues are dotted underlined.

Preferably, a LIM-like domain may comprise the amino acid sequence (SEQ ID NO: 29);

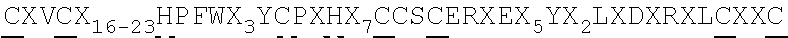

where X is any amino acid, Zn coordinating residues are solid underlined and putative Zn coordinating residues are dotted underlined.

More preferably, a LIM-like domain may comprise the amino acid sequence (SEQ ID NO: 30);

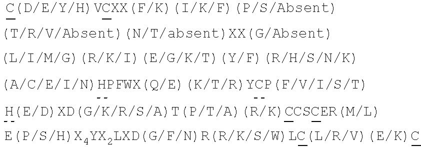

where X is any amino acid, Zn coordinating residues are solid underlined and putative Zn coordinating residues are dotted underlined.

In some embodiments, a LIM-like domain may comprise the amino acid sequence of the AtDA1 LIM-like domain;

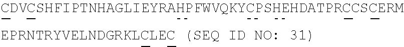

Other LIM-like domains include the LIM domain of an DA1 amino acid sequence shown in FIG. 5 (solid box), for example residues 200 to 266 of SEQ ID NO: 4 (Si_GI-514815267.pro), residues 182 to 248 of SEQ ID NO: 5 (Bd_GI-357157184.pro), residues 214 to 280 of SEQ ID NO: 6 (Br_DA1b.pro), residues 231 to 297 of SEQ ID NO: 7 (Br_DA1a.pro), residues 231 to 297 of SEQ ID NO: 8 (At_GI-15221983.pro), residues 176 to 242 of SEQ ID NO: 9 (Tc_GI-508722773.pro), residues 176 to 242 of SEQ ID NO: 10 (Gm_GI-356564241.pro), residues 180 to 246 of SEQ ID NO: 11 (Gm_GI-356552145.pro), residues 178 to 244 of SEQ ID NO: 12 (Vv_GI-302142429.pro), residues 181 to 247 of SEQ ID NO: 13 (Vv_GI-359492104.pro), residues 184 to 250 of SEQ ID NO: 14 (SI_GI-460385048.pro), residues 575 to 641 of SEQ ID NO: 15 (Os_GI-218197709.pro), residues 183 to 149 of SEQ ID NO: 16 (Os_GI-115466772.pro), residues 209 to 275 of SEQ ID NO: 17 (Bd_GI-357160893.pro), residues 191 to 257 of SEQ ID NO: 18 (Bd_GI-357164660.pro), residues 183 to 249 of SEQ ID NO: 19 (Sb_GI-242092232.pro), residues 206 to 272 of SEQ ID NO: 20 (Zm_GI-212275448.pro), residues 249 to 315 of SEQ ID NO: 21 (At_GI-240256211.pro), residues 221 to 287 of SEQ ID NO: 22 (At_GI-145360806.pro), residues 1298 to 1363 of SEQ ID NO: 23 (At_GI-22326876.pro), residues 130 to 176 of SEQ ID NO: 24 (At_GI-30698242.pro), residues 406 to 465 of SEQ ID NO: 25 (At_GI-30698240.pro), residues 345 to 404 of SEQ ID NO: 26 (At_GI-15240018.pro) or residues 256 to 319 of SEQ ID NO: 27 (At_GI-334188680.pro).

LIM-like domain sequences in other DA1 proteins may be identified using standard sequence analysis techniques using the the above information (e.g. Simple Modular Architecture Research Tool (SMART); EMBL Heidelberg, DE).

In addition to a LIM domain and a LIM-like domain, a DA1 protein may further comprise a carboxyl terminal region having an amino acid sequence at least 20%, at least 30%, at least 40%, at least 50%, at least 60%, at least 70%, at least 80%, at least 90%, at least 95%, or at least 98% amino acid identity to the sequence of residues 198 to 504 of SEQ ID NO: 4, residues 180 to 487 of SEQ ID NO: 5, residues 212 to 514 of SEQ ID NO: 6, residues 229 to 532 of SEQ ID NO: 7, residues 229 to 532 of SEQ ID NO: 8, residues 174 to 478 of SEQ ID NO: 9, residues 174 to 474 of SEQ ID NO: 10, residues 178 to 478 of SEQ ID NO: 11, residues 176 to 462 of SEQ ID NO: 12, residues 179 to 482 of SEQ ID NO: 13, residues 182 to 486 of SEQ ID NO: 14, residues 573 to 878 of SEQ ID NO: 15, residues 181 to 486 of SEQ ID NO: 16, residues 207 to 512 of SEQ ID NO: 17, residues 189 to 491 of SEQ ID NO: 18, residues 181 to 486 of SEQ ID NO: 19, residues 204 to 508 of SEQ ID NO: 20, residues 247 to 553 of SEQ ID NO: 21, residues 219 to 528 of SEQ ID NO: 22, residues 1296 to 1613 of SEQ ID NO: 23, residues 128 to 450 of SEQ ID NO: 24, residues 404 to 702 of SEQ ID NO: 25, residues 343 to 644 of SEQ ID NO: 26 or residues 256 to 587 of SEQ ID NO: 27.

The carboxyl terminal region of the DA1 protein may comprise the metallopeptidase active site motif HEMMH (SEQ ID NO: 32).

The carboxyl terminal region may further comprise a EK(X)₈R(X)₄SEEQ (SEQ ID NO: 33) or EK(X)₈R(X)₄SEQ (SEQ ID NO: 34) motif positioned between the LIM domain and HEMMH motif.

In addition to a LIM domain and a conserved carboxyl terminal region, a DA1 protein may comprise a UIM1 domain and a UIM2 domain. The UIM1 and UIM2 domains may be located between the N terminal and the LIM domain of the DA1 protein.

A UIM1 domain may consist of the sequence of SEQ ID NO: 35 and a UIM2 domain may consist of the sequence of SEQ ID NO: 36.
p—pLpbA1 pb.Sbp-.pp p (SEQ ID NO: 35)
p—pLpbA1 pb.Sbp-spp p (SEQ ID NO:36)
   wherein;
   p is a polar amino acid residue, for example, C, D, E, H, K, N, Q, R, S or T;
   b is a big amino acid residue, for example, E, F, H, I, K, L, M, Q, R, W or Y;
   s is a small amino acid residue, for example, A, C, D, G, N, P, S, T or V;
   l is an aliphatic amino acid residue, for example, I, L or V;
is absent or is any amino acid, and
is any amino acid.

Further examples of UIM1 and UIM2 domain sequences may be identified using standard sequence analysis techniques as described herein (e.g. Simple Modular Architecture Research Tool (SMART); EMBL Heidelberg, DE).

In some preferred embodiments, a DA1 protein may comprise;
a LIM domain of SEQ ID NO:1,
LIM like domain of SEQ ID NO: 28,
a C terminal region having at least 20% sequence identity to residues 229 to 532 of SEQ ID NO: 8 or the equivalent region of any one of SEQ NOS 4 to 7 or 9 to 27, as set out above and comprising a EK(X)₈R(X)₄SEEQ or EK(X)₈R(X)₄SEQ motif and a HEMMH motif,
a UIM domain of SEQ ID NO:35, and
a UIM domain of SEQ ID NO:36.

A DA1 protein may comprise an amino acid sequence of a plant DA1 protein shown in FIG. 5 (SEQ ID NOS: 4 to 27) or may be an allele or variant of one of these sequences which has DA1 activity.

For example, a DA1 protein may comprise the amino acid sequence of AtDA1, AtDAR1, AtDAR2, AtDAR3, AtDAR4, AtDAR5, AtDAR6, AtDAR7, BrDA1a, BrDA1b, BrDAR1, BrDAR2, BrDAR3-7, BrDAL1, BrDAL2, BrDAL3, OsDA1, OsDAR2, OsDAL3, OsDAL5, PpDAL1, PpDAL2, PpDAL3, PpDAL4, PpDAL5, PpDAL6, PpDAL7, PpDAL8, SmDAL1, SmDAL2 or ZmDA1 (ACR35367.1 GI: 238008664), preferably AtDA1, AtDAR1 BrDA1a, BrDA1b, OsDA1 or ZmDA1 or an allele or variant of one of these sequences.

In some preferred embodiments, a DA1 protein may comprise the amino acid sequence of AtDA1 (SEQ ID NO: 8; AT1G19270; NP_173361.1 GI: 15221983) or may be an allele or variant of this sequence which has DA1 activity.

Other DA1 protein sequences which include the characteristic features set out above and encoding DA1 nucleic acid sequences may be identified using standard sequence analysis tools in any plant species of interest.

A DA1 protein in a plant species of interest may have an amino acid sequence which is a variant of a DA1 protein reference amino acid sequence set out herein.

A DA1 protein which is a homologue or variant of a reference plant DA1sequence, such as any one of SEQ ID NOS: 4-27, may comprise an amino acid sequence having at least 20%, at least 30%, at least 40%, at least 50%, at least 60%, at least 70%, at least 80%, at least 90%, at least 95%, or at least 98% sequence identity to the reference sequence.

Particular amino acid sequence variants that occur in a plant species may differ from a reference sequence set out herein by insertion, addition, substitution or deletion of 1 amino acid, 2, 3, 4, 5-10, 10-20 20-30, 30-50, or more than 50 amino acids.

In some embodiments, a DA1 polypeptide which is a variant of AtDA1 sequence of any one of SEQ NOS: 4 to 27 may comprise a LIM domain having the sequence of SEQ ID NO: 3 and a LIM-like domain having the sequence of SEQ ID NO: 31.

A nucleic acid encoding a DA1 protein may comprise a nucleotide sequence set out in a database entry selected from the group consisting of NM_101785.3 GI: 42562170 (AtDA1); NM_001057237.1 GI: 115454202 (OsDA1); BT085014.1 GI: 238008663 (ZmDA1) or may be an allele or variant of one of these sequences which encodes an active DA1 protein.

In some preferred embodiments, a nucleic acid encoding a DA1 protein may comprise the nucleotide sequence of AtDA1 (NM_101785.3 GI: 42562170), ZmDA1 (BT085014.1 GI: 238008663), OsDA1 (NM_001057237.1 GI: 115454202) or may be an allele or variant of any one of these sequences which encodes a protein with DA1 activity.

A nucleic acid that encodes a DA1 protein in a plant species of interest may have a nucleotide sequence which is a variant of a DA1 reference nucleotide sequence set out herein.

DA1 polypeptides and encoding nucleic acids may be identified in plant species, in particular crop plants, such as wheat, barley, maize, rice, and another agricultural plants, using routine sequence analysis techniques.

For example, variant nucleotide sequence may be a homologue of a reference DA1 sequence set out herein, and may differ from the reference DA1 nucleotide sequence by one or more of addition, insertion, deletion or substitution of one or more nucleotides in the nucleic acid, for example 2, 3, 4, 5-10, 10-20 20-30, 30-50, or more than 50, leading to the addition, insertion, deletion or substitution of one or more amino acids in the encoded protein. Of course, changes to the nucleic acid that make no difference to the encoded amino acid sequence are included. A nucleic acid encoding a DA1 protein may comprise a sequence having at least 20% or at least 30% sequence identity with the reference nucleic acid sequence, preferably at least 40%, at least 50%, at least 60%, at least 65%, at least 70%, at least 80%, at least 90%, at least 95% or at least 98%. Sequence identity is described herein.

Sequence identity is commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Sequence comparison may be made over the full-length of the relevant sequence described herein.

A DA1 nucleotide sequence which is a variant of a reference DA1 nucleic acid sequence set out herein, may selectively hybridise under stringent conditions with this reference nucleic acid sequence or the complement thereof.

Stringent conditions include, e.g. for hybridization of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

An alternative, which may be particularly appropriate with plant nucleic acid preparations, is a solution of 5×SSPE (final 0.9 M NaCl, 0.05M sodium phosphate, 0.005M EDTA pH 7.7), 5×Denhardt's solution, 0.5% SDS, at 50° C. or 65° C. overnight. Washes may be performed in 0.2×SSC/0.1% SDS at 65° C. or at 50-60° C. in 1×SSC/0.1% SDS, as required.

DA1 proteins and encoding nucleic acids may be identified in plant species, in particular crop plants, such as wheat, barley, maize, rice, and another agricultural plants, using routine sequence analysis techniques and/or comparison with the reference sequences set out herein.

The LIM domain, the LIM-like domain or both the LIM domain and the LIM-like domain of a DA1 protein for use as described herein may be inactivated or disrupted (“LIM disrupted-DA1 protein”).

LIM domains and LIM-like domains are described in detail above and may be identified within any DA1 protein using standard sequence analysis techniques.

A DA1 protein with an inactivated or disrupted LIM domain or LIM-like domain may display aberrant, for example increased or activated, peptidase activity. For example, inactivation or disruption of the LIM domain or LIM-like domain may reduce or prevent the domain from interacting with the C terminal region of the DA1 protein and inhibiting DA1 peptidase activity.

In some embodiments, a DA1 protein with an inactivated or disrupted LIM domain or LIM-like domain may be display reduced stability in a plant cell following ubiquitinylation compared to wild-type DA1 protein.

A disrupted or inactivated LIM domain or LIM-like domain may be unable to coordinate Zn or form Zn finger motifs, such that the function of the domain is abolished i.e. the disrupted LIM or LIM-like domain is unable to mediate protein: protein interactions. For example, a disrupted LIM domain or LIM-like domain may be unable to interact intramolecularly with the C terminal region of the DA1 protein to inhibit peptidase activity.

An inactivated or disrupted LIM domain or LIM-like domain may comprise a sequence alteration or mutation which abolishes one or more Zn finger motifs in the LIM or LIM-like domain.

The amino acid sequence of a DA1 protein may be altered or mutated by insertion, substitution or deletion of one or more amino acids relative to the wild-type amino acid sequence in order to inactivate the LIM domain or LIM-like domain. For example, 1, 2, 3, 4, 5, 6, 7, 8, 9 or 10 or more amino acids may be altered, for example deleted or substituted, relative to the wild-type amino acid sequence. In some embodiments, 1 to 30, 1 to 20 or 1 to 10 residues may be altered.

Single amino acid substitutions within LIM domains and LIM-like domains are sufficient to elicit a LIM knockout phenotype. LIM domains, for example, may be inactivated by mutations in the Zn coordinating residues or other residues within the LIM domain (McIntosh et al (1998) Am J Human Genet 63 1651-16581; Clough et al (1999) Human mutation 14 459-465; Hamlington et al (2001) Human mutation 18 458-464; Taira et al., Nature 1994 372, 677-9; Agulnick et al., Nature 1996 384, 270-2). LIM-like domains may also be inactivated by mutations in the Zn coordinating residues (i.e. conserved Cys residues), or other residues within the LIM-like domain (Yang et al The Plant Journal, (2010), 63, 283-296).

Suitable inactivating or disrupting mutations are preferably within the LIM domain or LIM-like domain or adjacent thereto.

An inactivated or disrupted LIM domain or LIM-like domain may comprise a mutation of one or more Zn coordinating residues, or putative Zn coordinating residues, for example a cysteine or histidine residue in a CxxC or CXXH context, and/or a mutation of one or more non-Zn coordinating amino acid residues.

An inactivated or disrupted LIM domain may comprise a mutation at one or more of the first, second, third, fourth, fifth, sixth, seventh and eighth Zn coordinating residues in the LIM domain as shown in SEQ ID NO: 1 or SEQ ID NO: 2 above. For example, a inactivated or disrupted LIM domain may comprise a mutation at one or more of the cysteine residues in the CXXC motifs or a cysteine or histidine residue in the HXXC motif, for example the cysteine/histidine residues shown in positions 1, 4, 22, 25, 28, 31, 49 and 52 of SEQ ID NO: 3 and underlined in SEQ ID NOS 1 and 2 above. The LIM domain of said DA1 protein may comprise a mutation at one or more of the underlined residues of the DA1 LIM domain shown above, preferably C141, C144, H162, C165,C168, C171, H189 and C192 of the DA1 sequence of SEQ ID NO: 4, C123, C126, H144, C147, C150, C153, H171 and C174 of the DA1 sequence of SEQ ID NO: 5, C155, C158, H176, C179, C182, C185, H203 and C206 of the DA1 sequence of SEQ ID NO: 6, C172, C175, H193, C196, C199, C202, H220 and C223 of the DA1 sequence of SEQ ID NO: 7, C172, C175, H193, C196, C199, C202, H220 and C223 of the AtDA1 sequence of SEQ ID NO: 8, C117, C120, H138, C141, C144, C147, H165 and C168 of the DA1 sequence of SEQ ID NO: 9, C177, C180, H198, C201, C204, C207, H225 and C228 of the DA1 sequence of SEQ ID NO: 10, C121, C124, H142, C145, C148, C151, H169 and C172 of the DA1 sequence of SEQ ID NO: 11, C119, C122, H140, C143, C146, C149, H167 and C170 of the DA1 sequence of SEQ ID NO: 12, C122, C125, H143, C146, C149, C152, H170 and C173 of the DA1 sequence of SEQ ID NO: 13, C125, C128, H146, C149, C152, C155, H173 and C176 of the DA1 sequence of SEQ ID NO: 14, C516, C519, H537, C540, C543, C546, H564 and C567 of the DA1 sequence of SEQ ID NO: 15, C124, C127, H145, C148, C151, C154, H172 and C175 of the DA1 sequence of SEQ ID NO: 16, C150, C153, H171, C174, C177, C180, H198 and C201 of the DA1 sequence of SEQ ID NO: 17, C132, C135, H153, C156, C159, C162, H180 and C183 of the DA1 sequence of SEQ ID NO: 18, C124, C127, H145, C148, C151, C154, H172 and C175 of the DA1 sequence of SEQ ID NO: 19, C147, C150, H168, C172, C175, C178, H196 and C199 of the DA1 sequence of SEQ ID NO: 20, C190, C193, H211, C204, C207, C210, H228 and C231 of the DA1 sequence of SEQ ID NO: 21, C162, C165, H183, C186, C189, C192, H210 and C213 of the DA1 sequence of SEQ ID NO: 22, C1240, C1243, H1261, C1264, C1267, C1270, H1287 and C1290 of the DA1 sequence of SEQ ID NO: 23, C347, C350, H368, C371, C374, C377, H398 and C401 of the DA1 sequence of SEQ ID NO: 25, C286, C289, H307, C310, C313, C316, H337 and C340 of the DA1 sequence of SEQ ID NO: 26, C201, C204, H222, C225, C228, C231, H248 and C251 of the DA1 sequence of SEQ ID NO: 27, or the equivalent cysteine residues in other DA1 protein sequences.

For example, the LIM disrupted DA1 protein may have a C to Y, C to G or other substitution at one or more of these positions.

Zn coordinating residues within the LIM domain of a DA1 protein may be identified by standard sequence analysis. Cysteine and histidine residues equivalent to C172, C175, H193, C196, C199, C202, H220 and C223 of SEQ ID NO: 8 are sequence residues in the same sequence context in a different DA1 protein sequence and may be identified by standard sequence analysis, as shown in FIG. 5.

An inactivated or disrupted LIM domain may comprise a mutation at one or more non-Zn coordinating residues in the LIM domain as shown in SEQ ID NO:1 or SEQ ID NO: 2 above. A non-Zn coordinating residue may be located within 4 residues of a Zn coordinating residue in the LIM domain sequence or may be located 4 or more residues away from a Zn coordinating residue.

An inactivated or disrupted LIM-like domain may comprise a mutation at one or more of the first, second, third, fourth, fifth, sixth, seventh and eighth Zn coordinating residues or putative Zn coordinating residues in the LIM-like domain as shown in any one of SEQ ID NOS: 28 to 31 above. For example, a inactivated or disrupted LIM-like domain may comprise a mutation at one or more of the cysteine residues in the CXXC motifs or a cysteine or histidine residue in the CXXH motif, for example the cysteine/histidine residues shown in positions 1, 4, 29, 32, 40, 43, 63 or 66 of SEQ ID NO: 31 and underlined in SEQ ID NOS 28 to 31 above. Two of the three putative Zn coordinating residues H252, C260, H263 in the LIM-like domain are responsible for Zn coordination (i.e. H252 and C260; H252 and H263; or C260 and H263). The LIM-like domain of said DA1 protein may comprise a mutation at one or more of the underlined residues of the AtDA1 LIM-like domain shown above, preferably C232, C235, H252, C260, H263, C271, C274, C294 and/or C297 of the AtDA1 sequence of SEQ ID NO: 8, or the equivalent cysteine residues in other DA1 protein sequences. For example, the LIM disrupted DA1 protein may have a C to Y, C to G or other substitution at one or more of these positions.

Cysteine residues equivalent to C232, C235, H252, C260, H263, C271, C274, C294 and C297 of SEQ ID NO: 8 are sequence residues in the same sequence context in a different DA1 protein sequence and may be identified by standard sequence analysis, as shown in FIG. 5.

An inactivated or disrupted LIM-like domain may comprise a mutation at one or more residues in the LIM-like domain other than conserved cysteine or histidine residues as shown in SEQ ID NO:28 to SEQ ID NO:31 above. Suitable residues may be located within 4 residues of a conserved cysteine or histidine residue in the LIM-like domain sequence or may be located 4 or more residues away from a conserved cysteine or histidine residue.

Some preferred mutations include the conversion of a Zn coordinating residue in a LIM or LIM-like domain, such as cysteine or histidine, to a neutral amino acid, such as glycine.

Other mutations that disrupt Zn finger motifs and are suitable for abolishing LIM or LIM-like function in a DA1 protein will be readily apparent to the skilled person. Unlike mutations in other domains within the DA1 protein, LIM domain and LIM-like domain mutations destabilise the DA1 protein in the presence of its interacting partner EOD1 in the plant cell. Suitable LIM domain and LIM-like domain mutations may therefore be identified by determining the stability the mutant DA1 protein in the presence of EOD1 using standard experimental techniques. Reduce stability relative to the wild-type DA1 is indicative that a mutation disrupts the LIM or LIM-like domain.

A LIM-disrupted DA1 protein as described herein may comprise a conserved R residue located at a position in the DA1 amino acid sequence which is equivalent to position 358 of SEQ ID NO: 8 of A. thaliana DA1, position 333 of SEQ ID NO: 20 of the Z. mays DA1 or the equivalent position in another DA1 amino acid sequence, for example a DA1 sequence of FIG. 5 (conserved R residue shown by arrow). The conserved R residue that is located at a position in a DA1 amino acid sequence which is equivalent to position 358 of SEQ ID NO: 8 of A. thaliana DA1 or position 333 of the Z. mays DA1 of SEQ ID NO: 20 is located at the position within the DA1 amino acid sequence which corresponds to R333 of SEQ ID NO:20 and R358 of SEQ ID NO:8 i.e. it is in the same position relative to to the other motifs and domains of the DA1 protein. The conserved R residue is located between the LIM domain and the HEMMH (SEQ ID NO: 32) peptidase motif of the C terminal region and is completely conserved in the same sequence context in DA1 proteins. The conserved R residue may be contained in a EK(X)₈R(X)₄SEEQ (SEQ ID NO: 33) or EK(X)₈R(X)₄SEQ (SEQ ID NO: 34) motif within the C terminal region.

The data herein shows that the LIM domain and the LIM-like domain do not mediate DA1 homodimersation and a LIM disrupted-DA1 protein retains the ability to bind to wild-type DA1.

Expression of a LIM-disrupted DA1 protein in one or more cells of a plant reduces DA1 activity in the cells and enhances yield-related plant traits, such as seed or organ size (see for example Li et al (2008); WO2009/047525; Wang et al 2012) thereby increasing plant yield. A plant expressing a LIM-disrupted DA1 protein may have a da1-1 or a da1-1 like phenotype.

In some embodiments, a LIM-disrupted DA1 protein may be expressed from heterologous nucleic acid in the one or more plant cells.

The LIM-disrupted DA1 protein may be expressed in one or more cells of a plant by any convenient technique and suitable techniques are well-known in the art.

Nucleic acid encoding the LIM-disrupted DA1 protein may be recombinantly expressed in the same plant species or variety from which it was originally isolated or in a different plant species or variety (i.e. a heterologous plant).

Nucleic acids provided may be double-or single-stranded, cDNA or genomic DNA, or RNA. The nucleic acid may be wholly or partially synthetic, depending on design. Naturally, the skilled person will understand that where the nucleic acid includes RNA, reference to the sequence shown should be construed as reference to the RNA equivalent, with U substituted for T.

“Heterologous” indicates that the gene/sequence of nucleotides in question or a sequence regulating the gene/sequence in question, has been introduced into said cells of the plant or an ancestor thereof, using genetic engineering or recombinant means, i.e. by human intervention. Nucleotide sequences which are heterologous to a plant cell may be non-naturally occurring in cells of that type, variety or species (i.e. exogenous or foreign) or may be sequences which are non-naturally occurring in that sub-cellular or genomic environment of the cells or may be sequences which are non-naturally regulated in the cells i.e. operably linked to a non-natural regulatory element.

Nucleic acid encoding the LIM-disrupted DA1 protein may be operably linked to a heterologous regulatory sequence, such as a promoter, for example a constitutive, inducible, tissue-specific or developmental specific promoter as described above.

The nucleic acid encoding the LIM-disrupted DA1 protein may be contained on a nucleic acid construct or vector. The construct or vector is preferably suitable for transformation into and/or expression within a plant cell. A vector is, inter alia, any plasmid, cosmid, phage or Agrobacterium binary vector in double or single stranded linear or circular form, which may or may not be self-transmissible or mobilizable, and which can transform prokaryotic or eukaryotic host, in particular a plant host, either by integration into the cellular genome or exist extrachromasomally (e.g. autonomous replicating plasmid with an origin of replication).

Specifically included are shuttle vectors by which is meant a DNA vehicle capable, naturally or by design, of replication in two different organisms, which may be selected from Actinomyces and related species, bacteria and eukaryotic (e.g. higher plant, mammalia, yeast or fungal) cells.

A construct or vector comprising nucleic acid as described above need not include a promoter or other regulatory sequence, particularly if the vector is to be used to introduce the nucleic acid into cells for recombination into the genome.

Constructs and vectors may further comprise selectable genetic markers consisting of genes that confer selectable phenotypes such as resistance to antibiotics such as kanamycin, hygromycin, phosphinotricin, chlorsulfuron, methotrexate, gentamycin, spectinomycin, imidazolinones, glyphosate and d-amino acids.

Those skilled in the art can construct vectors and design protocols for recombinant gene expression, for example in a microbial or plant cell. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 3rd edition, Sambrook et al, 2001, Cold Spring Harbor Laboratory Press and Protocols in Molecular Biology, Second Edition, Ausubel et al. eds. John Wiley & Sons, 1992. Specific procedures and vectors previously used with wide success upon plants are described by Bevan, Nucl. Acids Res. (1984) 12, 8711-8721), and Guerineau and Mullineaux, (1993) Plant transformation and expression vectors. In: Plant Molecular Biology Labfax (Croy RRD ed) Oxford, BIOS Scientific Publishers, pp 121-148.

When introducing a chosen gene construct into a cell, certain considerations must be taken into account, well known to those skilled in the art. The nucleic acid to be inserted should be assembled within a construct that contains effective regulatory elements that will drive transcription. There must be available a method of transporting the construct into the cell. Once the construct is within the cell membrane, integration into the endogenous chromosomal material either will or will not occur. Finally, the target cell type is preferably such that cells can be regenerated into whole plants.

It is desirable to use a construct and transformation method which enhances expression of the nucleic acid encoding the LIM or LIM-like disrupted DA1 protein. Integration of a single copy of the gene into the genome of the plant cell may be beneficial to minimize gene silencing effects. Likewise, control of the complexity of integration may be beneficial in this regard. Of particular interest in this regard is transformation of plant cells utilizing a minimal gene expression construct according to, for example, EP Patent No. EP1407000B1, herein incorporated by reference for this purpose.

Techniques well known to those skilled in the art may be used to introduce nucleic acid constructs and vectors into plant cells to produce transgenic plants with the properties described herein.

Agrobacterium transformation is one method widely used by those skilled in the art to transform plant species. Production of stable, fertile transgenic plants is now routine in the art (see for example Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. (1990) Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; WO92/14828; Nilsson, O. et al (1992) Transgenic Research 1, 209-220).

Other methods, such as microprojectile or particle bombardment (U.S. Pat. No. 5,100,792, EP-A-444882, EP-A-434616), electroporation (EP 290395, WO 8706614), microinjection (WO 92/09696, WO 94/00583, EP 331083, EP 175966, Green et al. (1987) Plant Tissue and Cell Culture, Academic Press), direct DNA uptake (DE 4005152, WO 9012096, U.S. Pat. No. 4,684,611), liposome mediated DNA uptake (e.g. Freeman et al. Plant Cell Physiol. 29: 1353 (1984)) or the vortexing method (e.g. Kindle, PNAS U.S.A. 87: 1228 (1990d)) may be preferred where Agrobacterium transformation is inefficient or ineffective, for example in some gymnosperm species. Physical methods for the transformation of plant cells are reviewed in Oard, 1991, Biotech. Adv. 9: 1-11.

Alternatively, a combination of different techniques may be employed to enhance the efficiency of the transformation process, e.g. bombardment with Agrobacterium coated microparticles (EP-A-486234) or microprojectile bombardment to induce wounding followed by co-cultivation with Agrobacterium (EP-A-486233).

Following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

Following transformation, a plant cell that expresses the LIM-disrupted DA1 protein may be identified and/or selected. A plant may be regenerated from the plant cell.

In other embodiments, a mutation may be introduced into a nucleic acid sequence within the genome of a plant cell which encodes a DA1 protein, such that the nucleic acid encodes a LIM-disrupted DA1 protein. For example, a mutation may be introduce into the sequence encoding the LIM domain or LIM-like domain of the DA1 protein. A plant may then be regenerated from the mutated cell.

The nucleic acid encoding the DA1 protein may be mutated by insertion, substitution or deletion of one or more nucleotides relative to the wild-type nucleotide sequence. For example, 1, 2, 3, 4, 5, 6, 7, 8, 9 or 10 or more nucleotides may be altered relative to the wild-type nucleotide sequence in order to inactivated the encoded LIM or LIM-like domain. The mutations inactivate or knock out the LIM domain and/or the LIM-like domain and are preferably in the region of the nucleic acid sequence encoding the LIM domain or the LIM-like domain. Prefered mutations do not cause frameshifts.

Techniques for the mutagenesis, inactivation or knockout of target genes are well-known in the art (see for example In Vitro Mutagenesis Protocols; Methods in Molecular Biology (2nd edition) Ed Jeff Braman; Sambrook J et al. 2012. Molecular Cloning: A Laboratory Manual (4th Edition) CSH Press; Current Protocols in Molecular Biology; Ed Ausubel et al (2013) Wiley). In some embodiments, mutations may be introduced into a target DA1 coding sequence by genome editing techniques, for example RNA guided nuclease techniques such as CRISPR, Zinc-finger nucleases (ZFNs) and transactivator-like effector nucleases (TALENs) (Urnov, F.D. et al Nature reviews. Genetics 11, 636-646 (2010); Joung, J.K. et al. Nature reviews. Molecular cell biology 14, 49-55 (2013); Gasiunas, G. et al PNAS USA 109, E2579-2586 (2012); Cong, L. et al. Science 339, 819-823 (2013)).

A plant that expresses a LIM-disrupted DA1 protein as described above (i.e. a DA1 protein with an inactivated or disrupted LIM domain or LIM-like domain) may be sexually or asexually propagated or grown to produce off-spring or descendants. Off-spring or descendants of the plant regenerated from the one or more cells may be sexually or asexually propagated or grown. The plant or its off-spring or descendents may be crossed with other plants or with itself.

The plant or its off-spring or descendents may be tested for seed size, organ size and/or plant yield relative to controls.

A plant which expresses a LIM-disrupted DA1 protein as described herein may display increased seed and/or organ size relative to the controls and may have higher plant yields.

The effect of dominant-negative DA1 alleles on yield-associated traits in plants is increased in plants that are deficient in EOD1 expression or activity (Li et al (2008), WO2009/047525).

A LIM-disrupted DA1 protein may be expressed as described above in a plant that is deficient in EOD1 expression or activity.

EOD1 proteins are plant E3 ubiquitin ligases (Disch et al. (2006), Li et al (2008), WO2009/047525). EOD1 proteins comprise an EOD domain. A plant EOD domain may consist of the amino acid sequence of SEQ ID NO: 37;
(E/K)RCVICQ(L/M)(K/R/G/T/E)Y(K/R)(R/I)(G/K)(D/N/E)(R/Q/K/L)Q(I/M/V)( K/N/T/A)L(L/P)C(K/S)H(V/A) H(S/T/G/A)(E/Q/D/S/G)C(I/G/T/V)(S/T)(K/R)W L(G/T/S)INK(V/I/A/K)CP(V/I)C (SEQ ID NO: 37)

In some preferred embodiments, an EOD1 protein may comprise a EOD domain having an amino acid sequence of residues 150 to 192 of SEQ ID NO: 38, residues 187 to 229 of SEQ ID NO: 39, residues 192 to 234 of SEQ ID NO: 40, residues 189 to 231 of SEQ ID NO: 41, residues 194 to 236 of SEQ ID NO: 42, residues 194 to 236 of SEQ ID NO: 43, residues 194 to 236 of SEQ ID NO: 44, residues 195 to 237 of SEQ ID NO: 45, residues 189 to 231 of SEQ ID NO: 46, residues 195 to 237 of SEQ ID NO: 47, residues 195 to 237 of SEQ ID NO: 48, residues 195 to 237 of SEQ ID NO: 49, residues 218 to 260 of SEQ ID NO: 50, residues 196 to 238 of SEQ ID NO: 51, residues 197 to 239 of SEQ ID NO: 52, or residues 193 to 235 of SEQ ID NO: 53.

Further suitable EOD domain sequences may be identified using standard sequence analysis techniques as described herein (e.g. Simple Modular Architecture Research Tool (SMART); EMBL Heidelberg, DE).

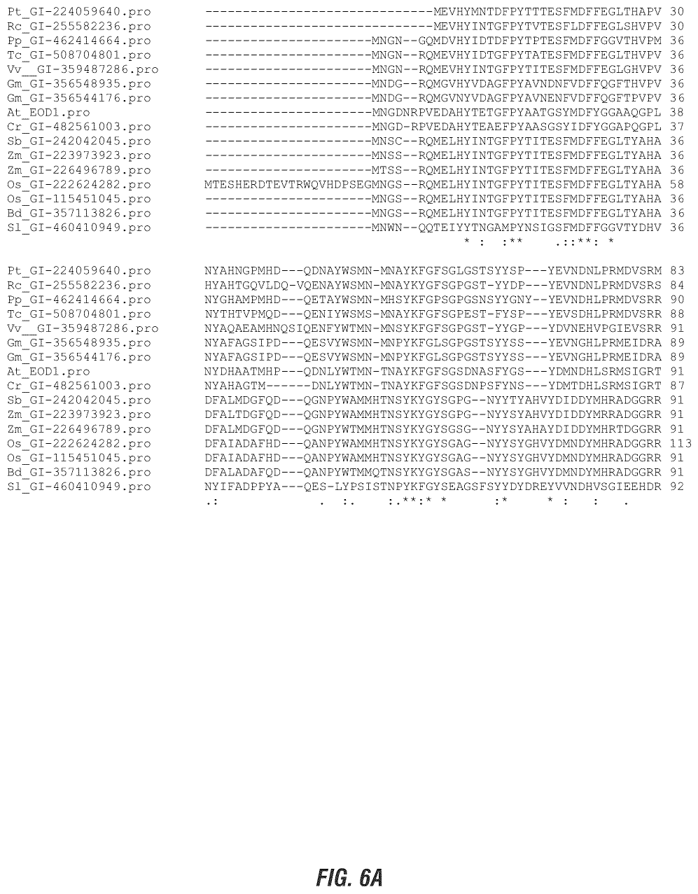

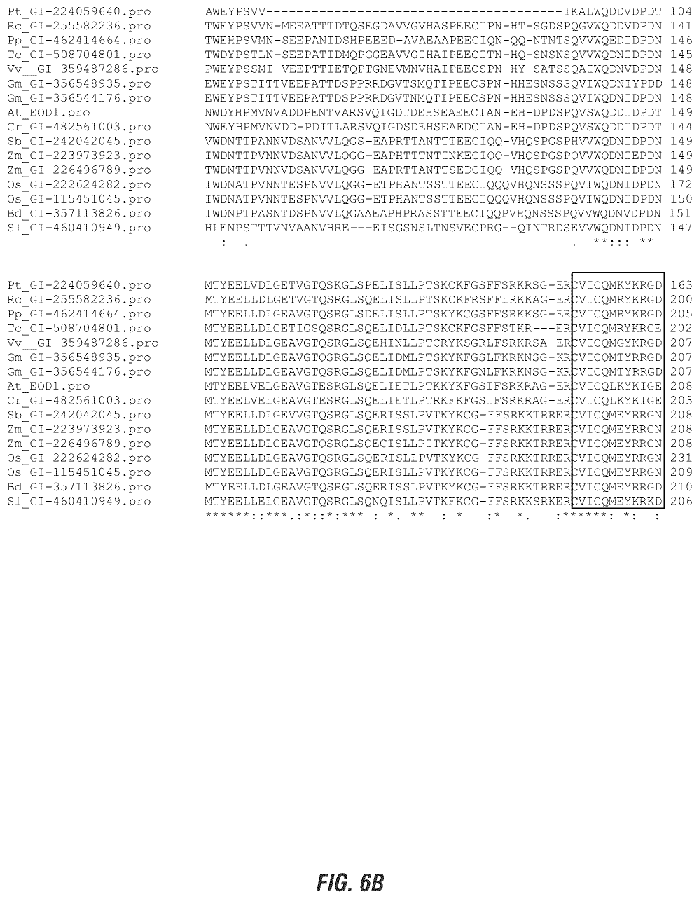

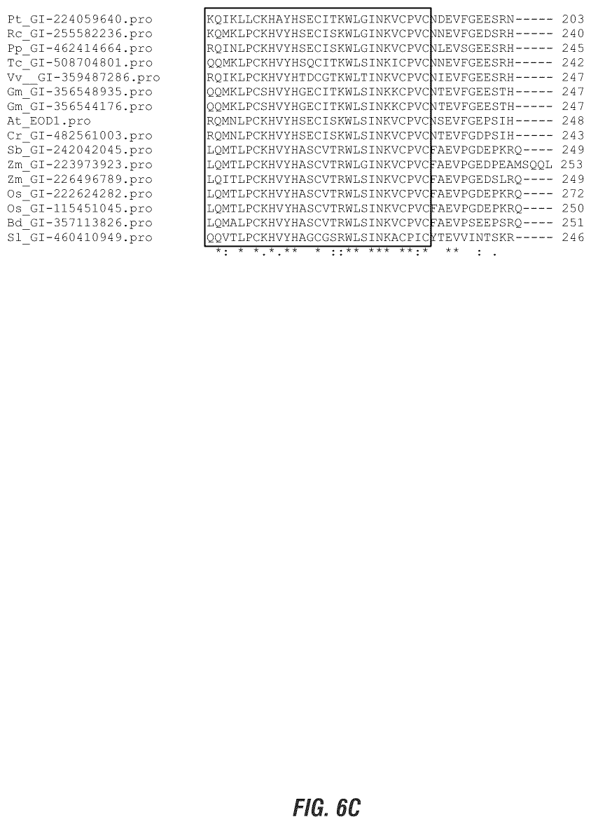

A EOD1 protein whose expression or activity is reduced in the plant cell expressing the LIM disrupted DA1 protein may comprise an amino acid sequence of any one of SEQ ID NOS 38 to 53 as set out in FIG. 6. In some preferred embodiments, a EOD1 protein may comprise the amino acid sequence of SEQ ID NO: 45 (AtEOD1) or SEQ ID NOS: 50 or 51 (OsEOD1) or may be a variant of this sequence which retains E3 ubiquitin ligase activity.

A EOD1 protein which is a variant of any one of SEQ ID NOS: 38 to 53 or other reference EOD1 sequence may comprise an amino acid sequence having at least 20%, at least 30%, at least 40%, at least 50%, at least 60%, at least 70%, at least 80%, at least 90%, at least 95%, or at least 98% sequence identity to the reference EOD1 sequence.

A EOD protein which is a variant of any one of SEQ ID NOS: 38 to 53 may further comprise a EOD domain having the sequence of SEQ ID NO: 37. Examples of suitable sequences are set out above.

A nucleic acid encoding a EOD1 protein may comprise a nucleotide sequence set out in a database entry selected from the group consisting of XM_002299911.1 GI: 224059639 (PtEOD1); XM_002531864.1 GI: 255582235 (RcEOD1); XM_002279758.2 GI: 359487285 (VvEOD1); XM_003542806.1 GI: 356548934 (GmEOD1a); XM_003540482.1 GI: 356544175 (GmEOD1b); XM_002468372.1 GI: 242042044 (SbEOD1); NM_001147247.1 GI: 226496788 (ZmEOD1); or NP_001030922.1 GI: 79316205 (AtEOD1; At3g63530) or may be variant of one of these sequences.

In some preferred embodiments, a nucleotide sequence encoding a EOD1 protein in a plant may encode AtEOD1 or OsEOD1 or may be a variant thereof.

EOD1 proteins and encoding nucleic acids whose expression or activity may be reduced as described herein may be readily identified in any plant species of interest, in particular a crop plant, such as wheat, barley, maize, rice, and another agricultural plants, using routine sequence analysis techniques.

Suitable methods for reducing EOD1 expression or activity are well-known in the art.

For example, the activity of EOD1 may be reduced, preferably abolished, by introducing a mutation, such as a deletion, insertion or substitution, at a position corresponding to position 44 of SEQ ID NO: 45, for example, an A to T substitution. A position in a EOD1 protein sequence which is equivalent to position 44 of SEQ ID NO: 45 may be identified using standard sequence analysis and alignment tools.

In some embodiments, the expression of a EOD1 protein may be reduced in a plant cell by expressing a heterologous nucleic acid which encodes or transcribes a suppressor nucleic acid, for example a suppressor RNA or RNAi molecule, within cells of said plant. The suppressor RNA suppresses the expression of EOD1 protein in the plant cells that express LIM-disrupted DA1.

A suitable RNAi sequence may correspond to a fragment of a reference EOD1 nucleotide sequence set out herein or may be a variant thereof.

In other embodiments, a knock out or knock down mutation may be introduced into a nucleic acid sequence within the genome of a plant cell which encodes an EOD1 protein, such that expression or activity of EOD1 is reduced. A plant may then be regenerated from the mutated cell.

The nucleic acid encoding EOD1 may be mutated by insertion, substitution or deletion of one or more nucleotides relative to the wild-type nucleotide sequence. For example, 1, 2, 3, 4, 5, 6, 7, 8, 9 or 10 or more nucleotides may be altered relative to the wild-type nucleotide sequence.

LIM-disrupted DA1 proteins may be expressed as described herein in any plant species. Examples of suitable plants for use in accordance with any aspect of the invention described herein include monocotyledonous and dicotelydonous higher plants, for example agricultural or crop plants, such as plants selected from the group consisting of Lithospermum erythrorhizon, Taxus spp, tobacco, cucurbits, carrot, vegetable brassica, melons, capsicums, grape vines, lettuce, strawberry, oilseed brassica, sugar beet, wheat, barley, maize, rice, soyabeans, peas, sorghum, sunflower, tomato, potato, pepper, chrysanthemum, carnation, linseed, hemp and rye.

Another aspect of the invention provides a transgenic plant which expresses a LIM-disrupted DA1 protein, as described above.

The plant may comprise an exogenous nucleic acid which encodes the LIM-disrupted DA1 protein.

One or more yield-related traits in the plant may be improved, increased or enhanced in the plant relative to control plants which do not express LIM-disrupted DA1 protein. Yield-related traits may include life-span, organ size and seed size.

The plant may have increased yield relative to control wild-type plants (i.e. identical plants which do not express a LIM-disrupted DA1 protein). For example, the mass of seeds (e.g. grain) or other plant product per unit area may be increased relative to control plants.

A suitable plant may be produced by a method described above.

In addition to a plant produced by a method described herein, the invention encompasses any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated off-spring, clone or descendant of such a plant, or any part or propagule of said plant, off-spring, clone or descendant.

A plant according to the present invention may be one which does not breed true in one or more properties. Plant varieties may be excluded, particularly registrable plant varieties according to Plant Breeders Rights.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

All documents mentioned in this specification are incorporated herein by reference in their entirety for all purposes.

The contents of all database entries mentioned in this specification are also incorporated herein by reference in their entirety for all purposes. This includes the versions of any sequences which are current at the filing date of this application.

### EXPERIMENTS

### 1. Methods

### 1.1 Co-Immunoprecipitation Analysis

All bait proteins for these studies were GST-tagged and glutathione sepharose beads (GE Life Science 17-0756-01) were used for their pull-down.

A flask of 10 ml LB with appropriate antibiotics was inoculated with a BL21 glycerol stock of the appropriate expression construct and left to grow overnight at 37° C. and 220 rpm. The following morning the 10 ml preculture was used to inoculate an 100 ml LB flask (at a ratio of 1:100), and this culture was incubated at 37° C. for two hours at 220 rpm. The flask was removed from the incubator, IPTG (Melford MB1008) was added to a final concentration of 1 mM before the culture was incubated at 28° C. (and 220 rpm) for another three hours.

Following this growth phase, the cultures were centrifuged at 4500×g for 10 minutes, the supernatants were discarded and the pellets resuspended at 4° C. in 2.5 ml TGH Buffer (50 mM HEPES (pH7.5), 150 mM NaCl, 1% Triton-X-100, 10% Glycerol, 1 mM DTT, 1 complete EDTA-free protease inhibitor tablet (per 50 ml) (Roche 11873580001)). The bacterial suspension was then sonicated (on ice) for four bursts of ten seconds, separated by 20-second intervals, before being centrifuged at 12 000×g for 20 minutes to pellet any cellular debris. Cleared sonicates were then stored on ice while a 50% slurry of washed glutathione sepharose beads (GE Life Sciences 17-0756-01) was prepared according to the manufacturer's instructions. 20 μl of the 50% glutathione sepharose slurry was then combined with 2.5 ml of protein extract from bait protein (GST-tagged) expressing cells and 2.5 ml of protein extract from prey protein (HA-/FLAG-/HIS-tagged) expressing cells. This mixture was incubated for 30 minutes at 4° C. on a rotating wheel and then the glutathione sepharose beads were washed five times with an excess (500 μl) of TGH buffer (following manufacturer's instructions). After washing, proteins were eluted with 35 μl GST-elution buffer (50 mM TRIS-glycine (pH8.0), 10 mM reduced glutathione) over 30 minutes at 4° C. before being analysed by western blot analysis.

### 1.2 Western Blots

20%, 12% or 4-20% precast SDS-polyacrylamide gels (RunBlue NXG02012, NXG01227, NXG42027) were submerged in RunBlue SDS-TRIS-tricine run buffer (RunBlue NXB0500), in a gel tank (Atto Japan AE6450) Samples were mixed with 2× Laemmli sample buffer (Bio-Rad Ltd 161-0737) placed in a heat block for 10 minutes at 96° C. and then loaded into rinsed wells in the gel in either 10 μl or 20 μl aliquots. The gels were run at 160V for 60 minutes along with a 3 ul aliquot of PageRuler Plus Prestained Protein Ladder, 10 to 250 kDa (Fermentas 26619). If appropriate, gels were stained at this stage.

Transfers were carried out using the Bio-Rad Mini Trans-Blot® Cell kit (Bio-Rad 170-3836). Gels were removed from their glass casing and laid on top of a sponge (from Bio-Rad Mini Trans-Blot® Cell kit), two pieces of chromatography paper (VWR WHAT3030-917) and a methanol-washed PVDF membrane (Roche Diagnostics 03010040001). Air bubbles were removed from between the gel and membrane and then two further pieces of Whatman paper and a sponge were applied to the gel. This was enclosed in a gel holder cassette (from Bio-Rad Mini Trans-Blot® Cell kit), submerged in transfer buffer (25 mM TRIS, 192 mM glycine, 10% (v/v) methanol) and run at 90V for 70 minutes at 4° C.

Following the transfer the membrane was washed for 10 minutes in 50 ml PBS (140 mM NaCl, 2.7 mM KCl, 10 mM Na₂HPO₄, 1.8 mM KH₂PO₄, pH 7.3) at room temperature, before being agitated in 50 ml blocking solution (5% (w/v) milk powder, 0.1% (v/v) Tween-20) for either one hour at room temperature or overnight at 4° C. Primary antibodies were diluted to their appropriate concentration in blocking solution and incubated with the membrane (10 ml per membrane with gentle agitation) for one hour before five washes with 50 ml PBST (140 mM NaCl, 2.7 mM KCl, 10 mM Na₂HPO₄, 1.8 mM KH₂PO₄, 0.1% (v/v) Tween-20,pH 7.3) at room temperature. If secondary antibody was required, staining and washing steps were repeated.

The washed membrane was held with forceps and carefully one corner was blotted onto blue-roll to remove excess moisture. It was then laid in a petri dish and treated with peroxidise substrate (SuperSignal West FEMTO Max. Sensitivity substrate (Fisher Scientific PN34095)) at a rate of 800 μl substrate per membrane. Membranes were left in this substrate for five minutes, dried as before and placed in an X-ray cassette under a piece of X-ray film (Fuji Film X-RAY 18×24 cm—(FujiFilm 497772RXNO)). X-ray films were developed using a Konica SRX-101 Table Top X-ray film developer (Konica 106931659).

Subsequent to analysis, if required, membranes were washed in 50 ml PBST and stained with 10 ml Ponceau S solution (Sigma-Aldrich P7170) for 30 minutes, followed by a single wash in 50 ml PBST and drying at room temperature.

### 1.3 Seed Size Determination

Seed area was used as a proxy measurement of seed size. Seeds were scattered in a petri dish and scanned against a white background using a desktop scanner (Hewlett Packard Scanjet 4370) at a high resolution (<3600 dpi). Images were stored as black and white 8-bit images and subjected to image analysis using the ImageJ software. ImageJ was opened and the threshold (Ctrl+Shift+T) set such that all seeds are completely red, then select all seeds with the “rectangular selection” tool and chose the analyse option (Analyze>Analyze Particles). In the dialog box set a size threshold to exclude smaller (non-seed) structures and large structures such as aggregations of seeds. Seed lengths and widths were calculated by fitting an ellipse to each seed (Analyze>Set measurements>Fit ellipse). When this option is selected the analysis outputs a “Major” and “Minor” value corresponding to length and width of the ellipse, representing the longest and widest parts of the seed. [J1]

### 2. Results

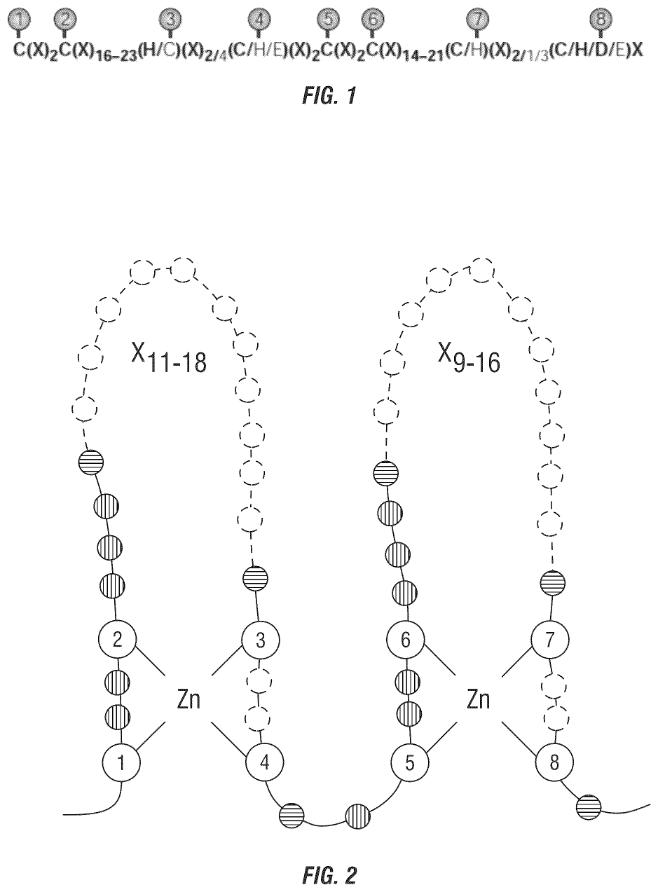

LIM domains (Prosite: PS00478) are a tandem zinc finger domains that act as a platform for protein: protein interactions (FIG. 1).

Web-based domain prediction software (Pfam, SMART, PROSITE) predicts the presence of a single LIM domain in DA1 (AtDA1 170aa-230aa), which was assumed to be involved in mediating putative DA1-DA1 homo-dimerisation (Li et al., 2008).

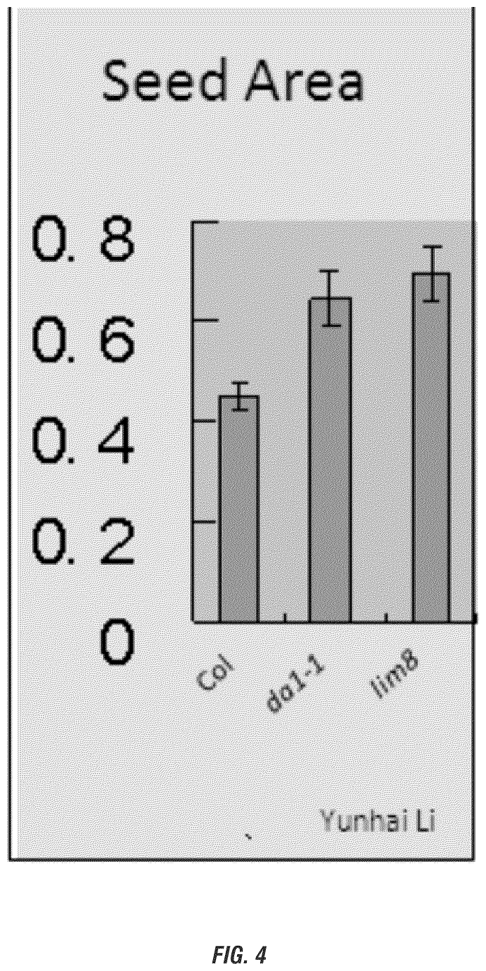

Surprisingly however, a variant of DA1 with a mutated LIM domain (henceforth ‘DA1lim8’) induced a dominant negative organ size phenotype equivalent to the da-1 mutant when introduced into a Col background in Arabidopsis (FIG. 4). This shows that the LIM domain of DA1 is not involved in DA1 homo-dimerisation.

4 key zinc coordinating amino acids (C172, C175, C199 and C202) were converted to glycines to produce the DA1lim8 mutant. These mutations were predicted to abolish the Zn finger motifs, which are due to Zn coordination by patterns of cysteine (C) residues.

Recombinant GST-tagged bait proteins were incubated with recombinant FLAG-tagged prey proteins before precipitation of GST-tagged bait proteins on glutathione sepharose beads. The purified proteins were then eluted and subjected to SDS-PAGE and immunoblot analysis. The ability of β-glucuronidase (GUS) to form a homo-tetramer was utilised to design a positive control of GST-GUS vs FLAG-GUS. Two sets of negative controls were also used; these were GST-GUS vs FLAG-prey, and GST-bait vs FLAG-GUS.

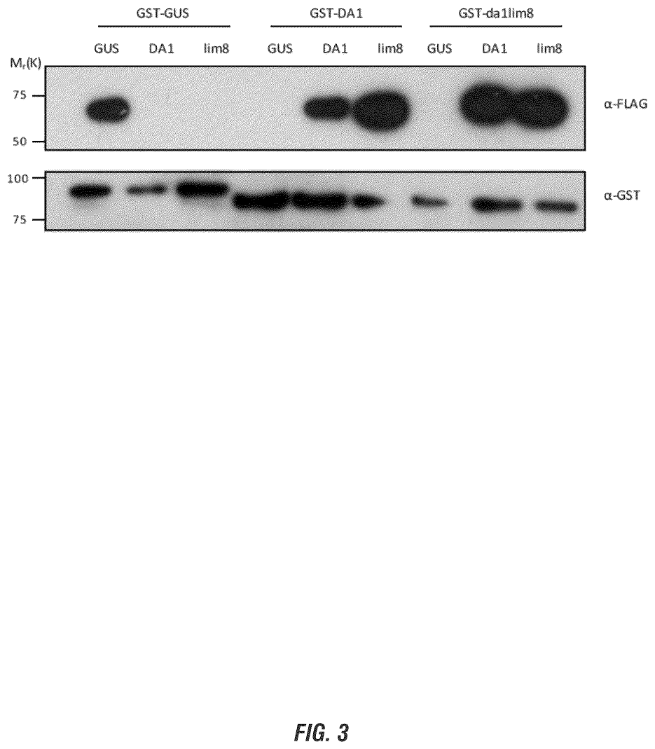

These in vitro co-immunoprecipitation experiments showed that Da1lim8 is able to bind to wild-type DA1 protein (FIG. 3) and that the lim8 seed size phenotype in a Col background is equivalent to that of da1-1 (FIG. 4).

The sequences of DA1 proteins were further analysed using a two-step domain prediction analysis. First, an initial homology detection screen (HHpred) was carried out to identify proteins with similar domains and structures. This was then followed by a domain prediction screen (Pfam, SMART, PROSITE), which used these proteins as query sequences. This strategy revealed that the region 230aa-297aa of AtDA1 shared significant structural homology with the LIM domains of other proteins (including the mouse LIM/homeobox protein LHX3). This new putative domain was termed the LIM-like domain.

The purported second pair of zinc coordinating amino acids in the LIM-like domain of DA1 was not detected by classical domain prediction software (Pfam, SMART, PROSITE) because of significant sequence divergence from the canonical LIM pattern. By considering a CxxH pairing at position 261aa-264aa in the AtDA1 sequence, it was apparent that an insertion in the first zinc finger domain and the inter-finger region causes the sequence to deviate significantly from the LIM consensus pattern. This results in a finger length of 24aa and an inter-finger region of 7aa (rather than 16-23aa and 2aa respectively).

The LIM-like domain therefore represents a second Zn finger containing LIM domain within the DA1 protein.

1. A method of increasing the yield of a plant or enhancing a yield-related trait in a plant comprising;
expressing a DA1 protein within cells of said plant,
wherein said the amino acid sequence of the DA1 protein comprises a mutation that disrupts or inactivates the LIM domain or the LIM-like domain of the DA1 protein.

2. A method according to claim 1 wherein the DA1 protein is expressed from a heterologous nucleic acid coding sequence in one or more cells of the plant.

3. A method of producing a plant with an increased yield and/or one or more enhanced yield-related traits comprising:
introducing into a plant cell a heterologous nucleic acid which encodes a DA1 protein,
wherein said the amino acid sequence of the DA1 protein comprises a mutation that disrupts or inactivates the LIM domain or the LIM-like domain of the DA1 protein, or
introducing a mutation into the nucleotide sequence of a plant cell which encodes a DA1 protein, such that the LIM domain or the LIM-like domain of the DA1 protein is disruped or inactivated, and
regenerating the plant from the plant cell.

4. A method according to claim 1 wherein the plant expressing the DA1 protein has increased life-span, organ size and/or seed size relative to controls.

5. A method according to claim 1 wherein the DA1 protein with the inactivated LIM and/or LIM-like domain has aberrant peptidase activity relative to the wild-type DA1 protein.

6. A method according to claim 1 wherein the LIM domain of the DA1 protein is inactivated.

7. A method according to claim 1 wherein the inactivated LIM domain of the DA1 protein comprises one or more sequence alterations relative to the wild-type LIM domain which inactivate LIM domain activity or function.

8. A method according to claim 6 wherein the wild-type LIM domain has the sequence of SEQ ID NO: 1 or SEQ ID NO: 2.

9. A method according to claim 8 wherein the wild-type LIM domain comprises two Zn finger motifs and the sequence alterations abolish one or both Zn finger motifs.

10. A method according to claim 9 wherein the sequence alterations include a mutation of one or more Zn coordinating residues in the LIM domain.

11. A method according to claim 10 wherein the sequence alterations include a mutation of one or more non-Zn coordinating residues in the LIM domain.

12. A method according to claim 11 wherein the non-Zn coordinating residues in the LIM domain are positioned within 4 residues of a Zn coordinating residue in the LIM domain.

13. A method according to claim 11 wherein the non-Zn coordinating residues in the LIM domain are positioned 4 or more residues from a Zn coordinating residue in the LIM domain.

14. A method according to claim 1 wherein the LIM-like domain of the DA1 protein is inactivated.

15. A method according to claim 14 wherein the inactivated LIM-like domain of the DA1 protein comprises one or more sequence alterations relative to the wild-type LIM-like domain which inactivate LIM-like domain activity or function.

16. A method according to claim 15 wherein the wild-type LIM-like domain has the sequence of SEQ ID NO: 28, 29, 30 or 31.

17. A method according to claim 16 wherein the wild-type LIM-like domain comprises two Zn finger motifs and the sequence alterations abolish one or both Zn finger motifs.

18. A method according to claim 17 wherein the sequence alterations include a mutation of one or more Zn coordinating residues in the LIM-like domain.

19. A method according to claim 16 wherein the sequence alterations include a mutation of one or more non-Zn coordinating residues in the LIM-like domain.

20. A method according to claim 19 wherein the non-Zn coordinating residues in the LIM-like domain are positioned within 4 residues of a Zn coordinating residue in the LIM domain.

21. A method according to claim 19 wherein the other residues in the LIM-like domain are positioned 4 or more residues from a conserved cysteine residues in the LIM-like domain.

22. A method according to claim 1 wherein the DA1 protein comprises a C terminal region having at least 20% sequence identity to residues 229 to 532 of SEQ ID NO: 8.

23. A method according to claim 22 wherein the C terminal region comprises the metallopeptidase motif HEMMH (SEQ ID NO: 32).

24. A method according to claim 23 wherein the C terminal region comprises the amino acid sequence EK(X)₈R(X)₄SEEQ (SEQ ID NO: 33) or EK(X)₈R(X)₄SEQ (SEQ ID NO: 34).

25. A method according to claim 1 wherein the DA1 protein comprises a UIM1 domain of SEQ ID NO: 35 and a UIM2 domain of SEQ ID NO: 36.

26. A method according to claim 1 wherein the DA1 protein comprises one or more sequence alterations relative to the wild-type DA1 sequence which disrupt or inactivate LIM domain activity or function.

27. A method according to claim 26 wherein the wild-type DA1 sequence comprises the amino acid sequence of any one of SEQ ID NOS: 4 to 27 or is a variant thereof.

28. A method according to claim 27 wherein the wild-type DA1 sequence comprises an amino acid sequence having at least 50% sequence identity to any one of SEQ ID NOS: 4 to 27.

29. A method according to claim 28 wherein the nucleic acid encoding the DA1 protein is operably linked to a heterologous promoter.

30. A method according to claim 29 wherein the promoter is a tissue-specific promoter or an inducible promoter.

31. A method according to claim 30 wherein the nucleic acid encoding the DA protein acid is comprised in one or more vectors.

32. A method according to claim 1 wherein the plant or plant cell is deficient in EOD1 expression or activity.

33. A method according to claim 1 comprising selecting a plant or plant cell having increased yield or one or more enhanced yield-related traits compared to control plants.

34. A method according to claim 1 comprising sexually or asexually propagating or growing off-spring or descendants of the plant expressing the DA1 protein.

35. A method according to claim 1 wherein the plant is a higher plant.

36. A method according to claim 35 wherein the plant is an agricultural plant selected from the group consisting of Lithospermum erythrorhizon, Taxus spp, tobacco, cucurbits, carrot, vegetable brassica, melons, capsicums, grape vines, lettuce, strawberry, oilseed brassica, sugar beet, wheat, barley, maize, rice, soyabeans, peas, sorghum, sunflower, tomato, potato, pepper, chrysanthemum, carnation, linseed, hemp and rye.

37. A plant cell comprising a heterologous nucleic acid encoding a DA1 protein having a disrupted or inactivated LIM domain.

38. A plant comprising a plant cell according to claim 37.

39. A plant according to claim 38 which is produced by a method according to claim 1.


SEQUENCE LISTING (54 sequences)

SEQ ID NO: 1 - protein, 66 aa, synthetic construct
  REGION 1..66: note = Synthetic sequence: LIM domain
  VARIANT 5..27: note = X is any amino acid and up to seven of them may be absent; represents a range of 16 - 23 amino acids
  VARIANT 28: note = X is His or Cys
  VARIANT 29..32: note = X is any amino acid and any two of them may be absent; represents a string of 2 or 4 amino acids.
  VARIANT 33: note = X is Cys, His or Glu
  VARIANT 40..60: note = X is any amino acid and up to seven of them may be absent; represents a range of 14 - 21 amino acids.
  VARIANT 61: note = X is Cys or His
  VARIANT 62..64: note = X is any amino acid and any one or two of them may be absent; represents a string of 2 or 1 or 3 amino acids.
  VARIANT 65: note = X is Cys, His, Asp or Glu
CXXCXXXXXXXXXXXXXXXXXXXXXXXXXXXXXXXCXXCXXXXXXXXXXXXXXXXXXXXXXXXXXX

SEQ ID NO: 2 - protein, 63 aa, synthetic construct
  REGION 1..63: note = Synthetic sequence: LIM domain
  VARIANT 5..27: note = X is any amino acid and up to seven of them may be absent; represents a range of 16 - 23 amino acids.
  VARIANT 38..58: note = X is any amino acid and up to seven of them may be absent; represents a range of 14 - 21 amino acids.
CXXCXXXXXXXXXXXXXXXXXXXXXXXHXXCXXCXXCXXXXXXXXXXXXXXXXXXXXXHXXCX

SEQ ID NO: 3 - protein, 53 aa, Arabidopsis thaliana
CAGCNMEIGHGRFLNCLNSLWHPECFRCYGCSQPISEYEFSTSGNYPFHKACY

SEQ ID NO: 4 - protein, 504 aa, Setaria italica
MGWLSKIFKGSVNRVSRGHYNGNSHEGYSTQHTKSYGAHGNEDEDMDHAIALSLSEQDQRKGKAIDTEHHLDEDEQLARALQENTSPTLDEDEQLARALQESMNDEHPPRQHIPIEDVHSESAPASSLPPYVFPTNGSRVCAGCKTPIGQGRFLSCMDSVWHPQCFRCYGCDIPISEYEFAVHEDHAYHRSCYKERFHPKCDVCNSFIPTNKNGLIEYRAHPFWMQKYCPSHENDGTPRCCSCERMEPKHSQYITLDDGRRLCLECLHTAIMDTNECQPLYIDIQEFYEGMNMKVEQQVPLLLVERQALNEAMEAEKIGHHLPETRGLCLSEEQIVRTILRRPIIGPGNRIIDMITGPYKLVRRCEVTAILILYGLPRLLTGSILAHEMMHAYLRLKGYRTLSPEVEEGICQVLAHLWLESEITSGSGSMATTSAASSSSSTSSSSKKGAKTEFEKRLGEFFKHQIETDPSVAYGDGFRAGMRAVERYGLRSTLDHIKLTGSFP

SEQ ID NO: 5 - protein, 487 aa, Brachypodium distachyon
MGWLNKIFKGSVNRVSRGNYDGNWHDGNSSENIRGAYDESDNEDIDRAIALSLAEEDPNKGKAIIDPDYSLEEDEQLARALHESLNTGSPPHQNVPVVDVPSERVPTREPPPPVFLSSGFRACAGCNNPIGNGRFLSCMDSVWHPQCFRCFACNKPISEYEFAMHENQPYHKSCYKDFFHPKCDVCKDFIPTNKDGLIEYRAHPFWMQKYCPSHEDDGTPRCCSCERMEPTDIKYIRLDDGRKLCLECLTSATMDSPECQHLYMDIQEFFEGLNMKVEQQVPLLLVERQALNEALEAEKSGHHLPETRGLCLSEEQIVRTILRRPTIGPGNRIIDMITGPYKLVRRCEVTAILILYGLPRLQTGSILAHEMMHAYLRLKGYRSLSPQVEEGICQVLSHMWLESEIIAGASGNTASTSVPSSSSAPTSSKKGAKTEFEKRLGAFIKNQIETDSSVEYGDGFRAGNRAVERYGLRSTLDHMKITGSFPY

SEQ ID NO: 6 - protein, 514 aa, Brassica rapa
MGWLNKIFKGSNQRHPLGNEHYHHNGGYYENYPHEHSEPSAETDADHTQEPSTSEEETWNGKENEEVDRVIALSILEEENQRPETNTGAWKHAMMDDDEQLARAIQESMIARNGTTYDFGNAYGNGHMHGGGNVYDNGDIYYPRPIAFSMDFRICAGCNMEIGHGRYLNCLNALWHPQCFRCYGCSHPISEYEFSTSGNYPFHKACYRERFHPKCDVCSLFISTNHAGLIEYRAHPFWVQKYCPSHEHDATPRCCSCERMEPRNTGYFELNDGRKLCLECLDSSVMDTFQCQPLYLQIQEFYEGLNMTVEQEVPLLLVERQALNEAREGERNGHYHMPETRGLCLSEEQTVRTVRKRSKGNWSGNMITEQFKLTRRCEVTAILILFGLPRLLTGSILAHEMMHAWMRLKGFRPLSQDVEEGICQVMAHKWLEAELAAGSRNSNAASSSSSSYGGVKKGPRSQYERKLGEFFKHQIESDASPVYGDGFRAGRLAVNKYGLWRTLEHIQMTGRFPV

SEQ ID NO: 7 - protein, 532 aa, Brassica rapa
MGWFNKIFKGSTQRFRLGNDHDHNGYYQSYPHDEPSADTDPDPDPDPDETHTQEPSTSEEDTSGQENEDIDRAIALSLIENSQGQTNNTCAANAGKYAMVDEDEQLARAIQESMVVGNTPRQKHGSSYDIGNAYGAGDVYGNGHMHGGGNVYANGDIYYPRPTAFPMDFRICAGCNMEIGHGRYLNCLNALWHPECFRCYGCRHPISEYEFSTSGNYPFHKACYRERYHPKCDVCSLFIPTNHAGLIGYRAHPFWVQKYCPSHEHDATPRCCSCERMEPRNTGYVELNDGRKLCLECLDSAVMDTFQCQPLYLQIQEFYEGLFMKVEQDVPLLLVERQALNEAREGEKNGHYHMPETRGLCLSEEQTVSTVRKRSKHGTGNWAGNMITEPYKLTRQCEVTAILILFGLPRLLTGSILAHEMMHAWMRLKGFRTLSQDVEEGICQVMAHKWLEAELAAGSRNSNVASSSSSRGVKKGPRSQYERKLGEFFKHQIESDASPVYGDGFRAGRLAVNKYGLPKTLEHIQMTGRFPV

SEQ ID NO: 8 - protein, 532 aa, Arabidopsis thaliana
MGWFNKIFKGSNQRLRVGNNKHNHNVYYDNYPTASHDDEPSAADTDADNDEPHHTQEPSTSEDNTSNDQENEDIDRAIALSLLEENQEQTSISGKYSMPVDEDEQLARALQESMVVGNSPRHKSGSTYDNGNAYGAGDLYGNGHMYGGGNVYANGDIYYPRPITFQMDFRICAGCNMEIGHGRFLNCLNSLWHPECFRCYGCSQPISEYEFSTSGNYPFHKACYRERYHPKCDVCSHFIPTNHAGLIEYRAHPFWVQKYCPSHEHDATPRCCSCERMEPRNTRYVELNDGRKLCLECLDSAVMDTMQCQPLYLQIQNFYEGLNMKVEQEVPLLLVERQALNEAREGEKNGHYHMPETRGLCLSEEQTVSTVRKRSKHGTGKWAGNITEPYKLTRQCEVTAILILFGLPRLLTGSILAHEMMHAWMRLKGFRTLSQDVEEGICQVMAHKWLDAELAAGSTNSNAASSSSSSQGLKKGPRSQYERKLGEFFKHQIESDASPVYGDGFRAGRLAVHKYGLRKTLEHIQMTGRFPV

SEQ ID NO: 9 - protein, 478 aa, Theobroma cacao
MDWIKKIFKGCAHKFSEGHHHGNYVEDPHPQFNAPSVSGDAWQELENEDVDRAIALSLLGESQKGRKVIDDEYQLEEDEQLARALQESLNFEPPPQYENANMYQPMPVHFPMGYRICAGCNTEIGHGRFLNCLNAFWHPECFRCHACNLPISDYEFSMSGNYRFHKSCYKERYHPKCDVCNDFIPTNPAGLIEYRAHPFWIQKYCPSHEHDSTPRCCSCERMEPQDTGYVALNDGRKLCLECLDSAVMDTKQCQPLYLDILEFYEGLNMKVEQQVPLLLVERQALNEAREGEKNGHYHMPETRGLCLSEEQTVSTILRQPRFGTGNRAMDMITEPCKLTRRCEVTAILILYGLPRLLTGSILAHEMMHAWMRLQGFRTLSQDVEEGICQVLAHMWLLTQLEYASSSNVASASSSASSRLQKGKRPQFEGKLGEFFKHQIESDTSPVYGDGFRAGHQAVYKYGLRRTLEHIRMTGRFPY

SEQ ID NO: 10 - protein, 474 aa, Glycine max
MGWLSRIFKGSDHNKLSEGHYYKEDAGYYLPSTSGVTNNQNENEDIDRAIALSLVEESRRANNNVNGERILSLQTLLEEDEQLARAIEQSLNLESPPRYGNENMYQPPIQYFPLGICAGCYTEIGFGRYLNCLNAFWHPECFRCRACNLPISDYEFSTSGNYPYHKSCYKESYHPKCDVCKHFIPTNPAGLIEYRAHPFWIQKYCPTHEHDGTPRCCSCERMESQEAGYIALKDGRKLCLECLDSSIMDTNECQPLHADIQRFYDSLNMKLDQQIPLLLVERQALNEAREGEKNGHYHMPETRGLCLSEELSTFSRRPRLGTAMDMRAQPYRPTTRCDVTAILVLYGLPRLLTGSILAHEMMHAWLRLKGYRTLSQDVEEGICQVLAHMWLESELSSASGSNFVSASSSSASHTSRKGKRPQFERKLGEFFKHQIESDISPVYGDGFRAGQKAVRKYGLQRTLHHIRMTGTFPY

SEQ ID NO: 11 - protein, 478 aa, Glycine max
MGWLSRIFKGSDHNKLSEGHYYKEDAGYYLPSTSGVTNDAWNQSQNQNENEDIDRAIALSLVEETQKANNNVNDYRSQLEEDEQLARAIEQSLNLESPPRYGNENMYQPPIQYFPMGSRICAGCYTEIGYGRYLNCLNAFWHPECFRCRACNLPISDYEFSTSGNYPYHKSCYKESYHPKCDVCKHFIPTNPAGLIEYRAHPFWIQKYCPTHEHDGTTRCCSCERMESQEAGYIALKDGRKLCLECLDSAIMDTNECQPLHADIQRFYESLNMKLDQQIPLLLVERQALNEAREGEKNGHYHMPETRGLCLSEELSTFSRRPRLGTTMDMRAQPYRPTTRCDVTAILILYGLPRLLTGSILAHEMMHAWLRLKGYRTLSQDVEEGICQVLSHMWLESELSSASGSNFVSASSSSASHTSRKGKRPQFERKLGEFFKHQIESDISPVYGGGFRAGQKAVSKYGLQRTLHHIRMTGTFPY

SEQ ID NO: 12 - protein, 462 aa, Vitis vinifera
MGWLNKIFKGSSHKISEGNYHGRYQGDTVQNEPSCSGDVWAETENEDIDRAIALSLSEEEQKGKKVIDNEFQLEEDEQLARAIQESLNIESPPQHGNGNGNGNIYQPIPFPYSTGFRICAGCNTEIGHGRFLSCMGAVWHPECFRCHGCGYPISDYEYSMNGNYPYHKSCYKEHYHPKCDVCKHFIPTNPAGLIEYRAHPFWVQKYCPSHEHDRTPRCCSCERMEPRDTRYVALNDGRKLCLECLDSAIMDTNECQPLYLDIQEFYEGLNMKVQQQVPLLLVERQALNEAMEGEKSGHHHMPETRGLCLSEEQTVSTILRRPKIGTGNRVMNMITEPCKLTRRCDVTAVLILYGLPRLLTGSILAHEMMHAWLRLNGYRTLAQDVEEGICQVLAYMWLDAELTSGSGRSQCERKLGQFFKHQIESDTSLVYGAGFRAGHQAVLKYGLPATLKHIHLTGNFPY

SEQ ID NO: 13 - protein, 482 aa, Vitis vinifera
MGWLNKIFKGSSHKISEGNYHGRYQGDTVQNEPSCSGDVWAETENEDIDRAIALSLSEEEQKGKKVIDELDNEFQLEEDEQLARAIQESLNIESPPQHGNGNGNGNIYQPIPFPYSTGFRICAGCNTEIGHGRFLSCMGAVWHPECFRCHGCGYPISDYEYSMNGNYPYHKSCYKEHYHPKCDVCKHFIPTNPAGLIEYRAHPFWVQKYCPSHEHDRTPRCCSCERMEPRDTRYVALNDGRKLCLECLDSAIMDTNECQPLYLDIQEFYEGLNMKVQQQVPLLLVERQALNEAMEGEKSGHHHMPETRGLCLSEEQTVSTILRRPKIGTGNRVMNMITEPCKLTRRCDVTAVLILYGLPRLLTGSILAHEMMHAWLRLNGYRTLAQDVEEGICQVLAYMWLDAELTSGSGSNVPSTSSASTSSKKGAGSQCERKLGQFFKHQIESDTSLVYGAGFRAGHQAVLKYGLPATLKHIHLTGNFPY

SEQ ID NO: 14 - protein, 486 aa, Solanum lycopersicum
MGWLNKIFRGSSHKISEGQYDWRCEGHTEEDDPSTAEDSWSEIEEIDRAIAISLSEEEQKGKIVIDSESQLKEDEQLARALQESLNVESPPQHVSRNDHGGGNVYGNGNFYHPVPFPYSASFRVCAGCSTEIGHGRFLSCMGAVWHPECFRCHACNQPISDYEFSMSGNYPYHKTCYKEHYHPKCDVCKHFIPTNAAGLIEYRAHPFWSQKYCPFHEHDGTPRCCSCERMEPRDTRYIALDDGRKLCLECLDSAIMDTSQCQPLYYDIQEFYEGLNMKVEQKVPLLLVERQALNEAMDGERHGYHHMPETRGLCLSEEQTISTIQRRPRIGAGNRVMDMRTEPYKLTRRCEVTAILILYGLPRLLTGSILAHEMMHAWLRLRGYRTLSQDVEEGICQVLAHMWLETQIASISSSNGGASTSSGMSSSKQGIRSPFERKLGDFFKHQIESDTSPIYGNGFRAGNQAVLKYGLERTLDHIRMTGTFPY

SEQ ID NO: 15 - protein, 878 aa, Oryza sativa
MGDRPDMGAGVALRFSHNDWTLEEDSKALHFLQPDLVLFTGDYGNENVQLVKSISDLQLPKAAILGNHDCWHTYQFSEKKVDRVQLQLESLGEQHVGYKCLDFPTIKLSVVGGRPFSCGGNRIFRPKLLSKWYGVNDMAESAKRIYDAATNAPKEHAVILLAHNGPTGLGSRMEDICGRDWVAGGGDHGDPDLEQAISDLQRETGVSIPLVVFGHMHKSLAYGRGLRKMIAFGANRTIYLNGAVVPRVNHAQSSRQPAISTSEKTGLEGLTGLMVPTSRAFTIVDLFEGAVEKISEVWVTVGDARTELEQELVLYKQPHKSVPSNIAIWSTMGWLTKFFRGSTHKISEGQYHSKPAEETIWNGPSNSAVVTMVYPLESTFGQLDLLLLATDLRQLVIDDVDCCKLRQQAQPVLHLMYSQLQLLQTSHAHQHGDVPSEFDNEDIARAISLSLLEEEQRKAKAIEKDMHLEEDEQLARAIQESLNVESPPRARENGNANGGNMYQPLPFMFSSGFRTCAGCHSEIGHGRFLSCMGAVWHPECFRCHACNQPIYDYEFSMSGNHPYHKTCYKERFHPKCDVCKQFIPTNMNGLIEYRAHPFWLQKYCPSHEVDGTPRCCSCERMEPRESRYVLLDDGRKLCLECLDSAVMDTSECQPLYLEIQEFYEGLNMKVEQQVPLLLVERQALNEAMEGEKTGHHHLPETRGLCLSEEQTVSTILRRPRMAGNKVMEMITEPYRLTRRCEVTAILILYGLPRLLTGSILAHEMMHAWLRLKGYRTLSPDVEEGICQVLAHMWIESEIIAGSGSNGASTSSSSSASTSSKKGGRSQFERKLGDFFKHQIESDTSMAYGDGFRAGNRAVLQYGLKRTLEHIRLTGTFPF

SEQ ID NO: 16 - protein, 486 aa, Oryza sativa
MGWLTKFFRGSTHKISEGQYHSKPAEETIWNGPSNSAVVTDVPSEFDNEDIARAISLSLLEEEQRKAKAIEKDMHLEEDEQLARAIQESLNVESPPRARENGNANGGNMYQPLPFMFSSGFRTCAGCHSEIGHGRFLSCMGAVWHPECFRCHACNQPIYDYEFSMSGNHPYHKTCYKERFHPKCDVCKQFIPTNMNGLIEYRAHPFWLQKYCPSHEVDGTPRCCSCERMEPRESRYVLLDDGRKLCLECLDSAVMDTSECQPLYLEIQEFYEGLNMKVEQQVPLLLVERQALNEAMEGEKTGHHHLPETRGLCLSEEQTVSTILRRPRMAGNKVMEMITEPYRLTRRCEVTAILILYGLPRLLTGSILAHEMMHAWLRLKGYRTLSPDVEEGICQVLAHMWIESEIIAGSGSNGASTSSSSSASTSSKKGGRSQFERKLGDFFKHQIESDTSMAYGDGFRAGNRAVLQYGLKRTLEHIRLTGTFPF

SEQ ID NO: 17 - protein, 512 aa, Brachypodium distachyon
MGWLTKIFRGSTYKISEGQRQSRPAEEAVWNEPSSSTVVTDVLSEFDNEDIDRAIALSLSEEQRKSKGTGKDLHLDEDEQLARAIHESLNVESPPCARDNGSPPHARDNSSPPHARENSSHPRARENGIANGGNSIQHSPFMFSSGFRTCAGCHSEIGHGRFLSCMGAVWHPECFCCHACSQPIYDYEFSMSGNHPYHKTCYKERFHPKCDVCKQFIPTNMNGLIEYRAHPFWLQKYCPSHEVDGTPRCCSCERMEPRESRYVLLDDGRKLCLECLDSAVMDTTECQPLYLEIQEFYEGLNMKVEQQVPLLLVERQALNEAMEGEKTGHHHLPETRGLCLSEEQTVSTILRRPRMTGNKIMEMITEPYRLTRRCEVTAILILYGLPRLLTGSILAHEMMHAWLRLKGYRTLSPEIEEGICQVLAHMWIESEIMAGSSSNAASTSSSSSSSISSKKGGRSQFERKLGDFFKHQIESDTSVAYGNGFRSGNQAVLQYGLKRTLEHIWLTGTWPF

SEQ ID NO: 18 - protein, 491 aa, Brachypodium distachyon
MGWLTKFFRGSTHNISEGQDQSKPAEETVWNEPSSSTAVNYALSEFDNEDIDRAIALSLSEEEQRKSKGTGKDQHLDEDEQLARAIQESLNVESPPRAREKSSHPRARENGSANGGNSYQLPLMFSSGFRTCAGCHSEIGHGRFLSCMGAVWHPECFCCHGCSQPIYDYEFSMSGNHPYHKTCYKERFHPKCDVCQQFIPTNTNGLIEYRAHPFWLQKYCPSHEVDGTPRCCSCERMEPRESRYVLLDDGRKLCLECLDSAVMDTTECQPLYLEIQEFYEGLNMKVEQQVPLLLVERQALNEAMEGEKTGHHHLPETRGLCLSEEQTVSTILRRPRMAGNKIMEMRTEPYRLTRRCEVTAILILYGLPRLLTGSILAHEMMHAWLRLKGYRTLSPDIEEGICQVLAHMWIESEITAGSGSNAASTSSSSTSSKKGGRSQFERKLGDFFKHQIESDTSVAYGDGFRAGNQAVLQYGLKRTLEHIRLTGTLPF

SEQ ID NO: 19 - protein, 486 aa, Sorghum bicolor
MGWLTKFFRGSTHNISEGQYHSRPAEDTAWNEPSSSPVVTDIFSEFNNEDIDRAIALSLSEEEQRKAKTIDKDMHLEEDEQLARAIQESLNVESPPPSRENGSANGGNAYHPLPFMFSSGFRACAGCHREIGHGRFLSCMGAVWHPECFRCHACSQPIYDYEFSMSGNHPYHKTCYKEQFHPKCDVCKQFIPTNMNGLIEYRAHPFWLQKYCPSHEVDGTPRCCSCERMEPRESRYVLLDDGRKLCLECLDSAVMDTNECQPLYLEIQEFYEGLNMKVEQQVPLLLVERQALNEAMEGEKAGHHHLPETRGLCLSEEQTVSTILRRPRMAGNKIMGMITEPYRLTRRCEVTAILILYGLPRLLTGSILAHEMMHAWLRLKGYRTLSPDVEEGICQVLAHLWIESEIMAGSGSGAASSSSGSSSSMSSKKAGRSQFEHKLGDFFKHQIETDTSMAYGEGFRAGNRAVLQYGLKRTLEHIRLTGTFPF

SEQ ID NO: 20 - protein, 508 aa, Zea mays
MGWLTKFFRGSTHNISEEQYHSRPAEDTAWNEPSSSPVVTDILSEFNNEDIDRAIALSLSEEEQRKEKAIDKDMHLEEDEQLARAIQESLNVESPPRRNGSANGGTMYHPPRETGNAYQPPRENGSANGGNAYHPLPFMFSSGFRACAGCHREIGHGRFLSCMGAVWHPECFRCHACSQPIYDYEFSMSGNHPYHKTCYKEQFHPKCDVCKQFIPTNMNGLIEYRAHPFWVQKYCPSHEMDGTPRCCSCERMEPRESKYVLLDDGRKLCLECLDSAVMDTNDCQPLYLEIQEFYEGLNMKVEQQVPLLLVERQALNEAMEGEKAGHHHLPETRGLCLSEEQTVSTILRPRMAGNKIMGMITEPYRLTRRCEVTAILILYGLPRLLTGSILAHEMMHAWLRLKGYRTLSPDVEEGICQVLAHMWIESEIMAGSGSSAASSSSGSSSSTSSKKGGRSQFEHRLGDFFKHQIETDTSMAYGDGFRTGNRAVLHYGLKRTLEHIRLTGTFPF

SEQ ID NO: 21 - protein, 553 aa, Arabidopsis thaliana
MGWLTKILKGSSHKFSDGQCNGRYREDRNLEGPRYSAEGSDFDKEEIECAIALSLSEQEHVIPQDDKGKKIIEYKSETEEDDDDDEDEDEEYMRAQLEAAEEEERRVAQAQIEEEEKRRAEAQLEETEKLLAKARLEEEEMRRSKAQLEEDELLAKALQESMNVGSPPRYDPGNILQPYPFLIPSSHRICVGCQAEIGHGRFLSCMGGVWHPECFCCNACDKPIIDYEFSMSGNRPYHKLCYKEQHHPKCDVCHNFIPTNPAGLIEYRAHPFWMQKYCPSHERDGTPRCCSCERMEPKDTKYLILDDGRKLCLECLDSAIMDTHECQPLYLEIREFYEGLHMKVEQQIPMLLVERSALNEAMEGEKHGHHHLPETRGLCLSEEQTVTTVLRRPRIGAGYKLIDMITEPCRLIRRCEVTAILILYGLPRLLTGSILAHEMMHAWLRLNGYPNLRPEVEEGICQVLAHMWLESETYAGSTLVDIASSSSSAVVSASSKKGERSDFEKKLGEFFKHQIESDSSSAYGDGFRQGNQAVLKHGLRRTLDHIRLTGTFP

SEQ ID NO: 22 - protein, 528 aa, Arabidopsis thaliana
MDSSSSSSSSSPSSSYGVARVSHISNPCIFGEVGSSSSSTYRDKKWKLMKWVSKLFKSGSNGGGSGAHTNHHPPQFQEDENMVFPLPPSSLDDRSRGARDKEELDRSISLSLADNTKRPHGYGWSMDNNRDFPRPFHGGLNPSSFIPPYEPSYQYRRRQRICGGCNSDIGSGNYLGCMGTFFHPECFRCHSCGYAITEHEFSLSGTKPYHKLCFKELTHPKCEVCHHFIPTNDAGLIEYRCHPFWNQKYCPSHEYDKTARCCSCERLESWDVRYYTLEDGRSLCLECMETAITDTGECQPLYHAIRDYYEGMYMKLDQQIPMLLVQREALNDAIVGEKNGYHHMPETRGLCLSEEQTVTSVLRRPRLGAHRLVGMRTQPQRLTRKCEVTAILVLYGLPRLLTGAILAHELMHGWLRLNGFRNLNPEVEEGICQVLSYMWLESEVLSDPSTRNLPSTSSVATSSSSSFSNKKGGKSNVEKKLGEFFKHQIAHDASPAYGGGFRAANAAACKYGLRRTLDHIRLTGTFPL

SEQ ID NO: 23 - protein, 1613 aa, Arabidopsis thaliana
MEPPAARVTPSIKADCSHSVNIICEETVLHSLVSHLSAALRREGISVFVDACGLQETKFFSIKQNQPLTDGARVLVVVISDEVEFYDPWFPKFLKVIQGWQNNGHVVVPVFYGVDSLTRVYGWANSWLEAEKLTSHQSKILSNNVLTDSELVEEIVRDVYGKLYPAERVGIYARLLEIEKLLYKQHRDIRSIGIWGMPGIGKTTLAKAVFNHMSTDYDASCFIENFDEAFHKEGLHRLLKERIGKILKDEFDIESSYIMRPTLHRDKLYDKRILVVLDDVRDSLAAESFLKRLDWFGSGSLIIITSVDKQVFAFCQINQIYTVQGLNVHEALQLFSQSVFGINEPEQNDRKLSMKVIDYVNGNPLALSIYGRELMGKKSEMETAFFELKHCPPLKIQDVLKNAYSALSDNEKNIVLDIAFFFKGETVNYVMQLLEESHYFPRLAIDVLVDKCVLTISENTVQMNNLIQDTCQEIFNGEIETCTRMWEPSRIRYLLEYDELEGSGETKAMPKSGLVAEHIESIFLDTSNVKFDVKHDAFKNMFNLKFLKIYNSCSKYISGLNFPKGLDSLPYELRLLHWENYPLQSLPQDFDFGHLVKLSMPYSQLHKLGTRVKDLVMLKRLILSHSLQLVECDILIYAQNIELIDLQGCTGLQRFPDTSQLQNLRVVNLSGCTEIKCFSGVPPNIEELHLQGTRIREIPIFNATHPPKVKLDRKKLWNLLENFSDVEHIDLECVTNLATVTSNNHVMGKLVCLNMKYCSNLRGLPDMVSLESLKVLYLSGCSELEKIMGFPRNLKKLYVGGTAIRELPQLPNSLEFLNAHGCKHLKSINLDFEQLPRHFIFSNCYRFSSQVIAEFVEKGLVASLARAKQEELIKAPEVIICIPMDTRQRSSFRLQAGRNAMTDLVPWMQKPISGFSMSVVVSFQDDYHNDVGLRIRCVGTWKTWNNQPDRIVERFFQCWAPTEAPKVVADHIFVLYDTKMHPSDSEENHISMWAHEVKFEFHTVSGENNPLGASCKVTECGVEVITAATGDTSVSGIIRESETITIIEKEDTIIDEEDTPLLSRKPEETNRSRSSSELQKLSSTSSKVRSKGNVFWKWLGCFPLQPKNLRSRSRRTTALEEALEEALKEREKLEDTRELQIALIESKKIKKIKQADERDQIKHADEREQRKHSKDHEEEEIESNEKEERRHSKDYVIEELVLKGKGKRKQLDDDKADEKEQIKHSKDHVEEEVNPPLSKCKDCKSAIEDGISINAYGSVWHPQCFCCLRCREPIAMNEISDLRGMYHKPCYKELRHPNCYVCEKKIPRTAEGLKYHEHPFWMETYCPSHDGDGTPKCCSCERLEHCGTQYVMLADFRWLCRECMDSAIMDSDECQPLHFEIREFFEGLHMKIEEEFPVYLVEKNALNKAEKEEKIDKQGDQCLMVVRGICLSEEQIVTSVSQGVRRMLNKQILDTVTESQRVVRKCEVTAILILYGLPRLLTGYILAHEMMHAYLRLNGYRNLNMVLEEGLCQVLGYMWLECQTYVFDTATIASSSSSSRTPLSTTTSKKVDPSDFEKRLVNFCKHQIETDESPFFGDGFRKVNKMMASNNHSLKDTLKEIISISKTPQYSKL

SEQ ID NO: 24 - protein, 450 aa, Arabidopsis thaliana
MVRRKRQEEDEKIEIERVKEESLKLAKQAEEKRRLEESKEQGKRIQVDDDQLAKTTSKDKGQINHSKDVVEEDVNPPPSIDGKSEIGDGTSVNPRCLCCFHCHRPFVMHEILKKGKFHIDCYKEYYRNRNCYVCQQKIPVNAEGIRKFSEHPFWKEKYCPIHDEDGTAKCCSCERLEPRGTNYVMLGDFRWLCIECMGSAVMDTNEVQPLHFEIREFFEGLFLKVDKEFALLLVEKQALNKAEEEEKIDYHRAAVTRGLCMSEEQIVPSIIKGPRMGPDNQLITDIVTESQRVSGFEVTGILIIYGLPRLLTGYILAHEMMHAWLRLNGYKNLKLELEEGLCQALGLRWLESQTFASTDAAAAAAVASSSSFSSSTAPPAAITSKKSDDWSIFEKKLVEFCMNQIKEDDSPVYGLGFKQVYEMMVSNNYNIKDTLKDIVSASNATPDSTV

SEQ ID NO: 25 - protein, 702 aa, Arabidopsis thaliana
MPISDVASLVGGAALGAPLSEIFKLVIEEAKKVKDFKPLSQDLASTMERLVPIFNEIDMMQQGSNRGTSELKVLTETMERAGEMVHKCSRIQWYSIAKKALYTREIKAINQDFLKFCQIELQLIQHRNQLQYMRSMGMASVSTKADLLSDIGNEFSKLCLVAQPEVVTKFWLKRPLMELKKMLFEDGVVTVVVSAPYALGKTTLVTKLCHDADVKEKFKQIFFISVSKFPNVRLIGHKLLEHIGCKANEYENDLDAMLYIQQLLKQLGRNGSILLVLDDVWAEEESLLQKFLIQLPDYKILVTSRFEFTSFGPTFHLKPLIDDEVECRDEIEENEKLPEVNPPLSMCGGCNSAVKHEESVNILGVLWHPGCFCCRSCDKPIAIHELENHVSNSRGKFHKSCYERYCYVCKEKKMKTYNIHPFWEERYCPVHEADGTPKCCSCERLEPRGTKYGKLSDGRWLCLECGKSAMDSDECQPLYFDMRDFFESLNMKIEKEFPLILVRKELLNKKEEKIDNHYEVLIRAYCMSEQKIMTYVSEEPRTGQNKQLIDMDTEPQGVVHECKVTAILILYGLPRLLTGYILAHEMMHAWLRLNGHMNLNNILEEGICQVLGHLWLESQTYATADTTADAASASSSSSRTPPAASASKKGEWSDFDKKLVEFCKNQIETDESPVYGLGFRTVNEMVTNSSLQETLKEILRRR

SEQ ID NO: 26 - protein, 644 aa, Arabidopsis thaliana
MASDYYSSDDEGFGEKVGLIGEKDRFEAETIHVIEVSQHEADIQKAKQRSLATHEAEKLDLATHEAEQLDLAIQEFSRQEEEEERRRTRELENDAQIANVLQHEERERLINKKTALEDEEDELLARTLEESLKENNRRKMFEEQVNKDEQLALIVQESLNMEEYPIRLEEYKSISRRAPLDVDEQFAKAVKESLKNKGKGKQFEDEQVKKDEQLALIVQESLNMVESPPRLEENNNISTRAPVDEDEQLAKAVEESLKGKGQIKQSKDEVEGDGMLLELNPPPSLCGGCNFAVEHGGSVNILGVLWHPGCFCCRACHKPIAIHDIENHVSNSRGKFHKSCYERYCYVCKEKKMKTYNNHPFWEERYCPVHEADGTPKCCSCERLEPRESNYVMLADGRWLCLECMNSAVMDSDECQPLHFDMRDFFEGLNMKIEKEFPFLLVEKQALNKAEKEEKIDYQYEVVTRGICLSEEQIVDSVSQRPVRGPNNKLVGMATESQKVTRECEVTAILILYGLPRLLTGYILAHEMMHAYLRLNGHRNLNNILEEGICQVLGHLWLDSQTYATADATADASSSASSSSRTPPAASASKKGEWSDFDKKLVEFCKNQIETDDSPVYGLGFRTVNEMVTNSSLQETLKEILRQR

SEQ ID NO: 27 - protein, 587 aa, Arabidopsis thaliana
MWCLSCFKPSTKHDPSEDRFEEETNIVTGISLYEDVILRQRRSEADQIEWAIQDSFNPQETSRCRQREEDDQIARGLQYVEETELDKSVVDEEDQQLSKIVEESLKEKGKSKQFEDDQVENDEQQALMVQESLYMVELSAQLEEDKNISTIPPLNEDAQLQKVIWESAKGKGQIEHFKDPVEEDGNLPRVDLNVNHPHSICDGCKSAIEYGRSVHALGVNWHPECFCCRYCDKPIAMHEFSNTKGRCHITCYERSHPNCHVCKKKFPGRKYKEHPFWKEKYCPFHEVDGTPKCCSCERLEPWGTKYVMLADNRWLCVKCMECAVMDTYECQPLHFEIREFFGSLNMKVEKEFPLLLVEKEALKKAEAQEKIDNQHGVVTRGICLSEGQIVNSVFKKPTMGPNGELVSLGTEPQKVVGGCEVTAILILYGLPRLLTGYILAHEMMHAWLRLNGTTSTQFVFANQYGESSQLKVLFGLITGYRNLKLELEEGICQVLGHMWLESQTYSSSAAASSASSSSRTPAANASKKGAQSDYEKKLVEFCKDQIETDDSPVYGVGFRKVNQMVSDSSLHKILKSIQHWTKPDSNL

SEQ ID NO: 28 - protein, 73 aa, synthetic construct
  REGION 1..73: note = Synthetic sequence: LIM-like domain
  VARIANT 5..27: note = X is any amino acid and up to seven of them may be absent; represents a range of 16 - 23 amino acids.
CXXCXXXXXXXXXXXXXXXXXXXXXXXHXXXXXXXCXXHXXXXXXXCXXCXXXXXXXXXXXXXXXXXXXCXXC

SEQ ID NO: 29 - protein, 73 aa, synthetic construct
  REGION 1..73: note = Synthetic sequence: LIM-like domain
  VARIANT 5..27: note = X is any amino acid and up to seven of them may be absent; represents a range of 16 - 23 amino acids.
CXVCXXXXXXXXXXXXXXXXXXXXXXXHPFWXXXYCPXHXXXXXXXCCSCERXEXXXXXYXXLXDXRXLCXXC

SEQ ID NO: 30 - protein, 66 aa, synthetic construct
  REGION 1..66: note = Synthetic sequence: LIM-like domain
  VARIANT 2: note = X is Asp, Glu, Tyr or His
  VARIANT 7: note = X is Phe or Lys
  VARIANT 8: note = X is Ile, Lys or Phe
  VARIANT 9: note = May be present or absent. If present, X is Pro or Ser
  VARIANT 10: note = May be present or absent. If present, X is Thr, Arg or Val
  VARIANT 11: note = May be present or absent. If present, X is Asn or Thr
  VARIANT 14: note = May be present or absent. If present, X is Gly
  VARIANT 15: note = X is Leu, Ile, Met or Gly
  VARIANT 16: note = X is Arg, Lys or Ile
  VARIANT 17: note = X is Glu, Gly, Lys or Thr
  VARIANT 18: note = X is Tyr or Phe
  VARIANT 19: note = X is Arg, His, Ser, Asn or Lys
  VARIANT 20: note = X is Ala, Cys, Glu, Ile or Asn
  VARIANT 26: note = X is Gln or Glu
  VARIANT 27: note = X is Lys, Thr or Arg
  VARIANT 31: note = X is Phe, Val, Ile, Ser or Thr
  VARIANT 33: note = X is Glu or Asp
  VARIANT 36: note = X is Gly, Lys, Arg, Ser or Ala
  VARIANT 38: note = X is Pro, Thr or Ala
  VARIANT 39: note = X is Arg or Lys
  VARIANT 46: note = X is Met or Leu
  VARIANT 48: note = X is Pro, Ser or His
  VARIANT 59: note = X is Gly, Phe or Asn
  VARIANT 61: note = X is Arg, Lys, Ser or Trp
  VARIANT 64: note = X is Leu, Arg or Val
  VARIANT 65: note = X is Glu or Lys
CXVCXXXXXXXXXXXXXXXXHPFWXXXYCPXHXXDXTXXCCSCERXEXXXXXYXXLXDXRXLCXXC

SEQ ID NO: 31 - protein, 66 aa, Arabidopsis thaliana
CDVCSHFIPTNHAGLIEYRAHPFWVQKYCPSHEHDATPRCCSCERMEPRNTRYVELNDGRKLCLEC

SEQ ID NO: 32 - protein, 5 aa, synthetic construct
  REGION 1..5: note = Synthetic sequence: Metallopeptidase active site motif
HEMMH

SEQ ID NO: 33 - protein, 19 aa, synthetic construct
  REGION 1..19: note = Synthetic sequence: Carboxyl terminal region motif
EKXXXXXXXXRXXXXSEEQ

SEQ ID NO: 34 - protein, 18 aa, synthetic construct
  REGION 1..18: note = Synthetic sequence: Carboxyl terminal region motif
EKXXXXXXXXRXXXXSEQ

SEQ ID NO: 35-36 - skipped (2 entries)

SEQ ID NO: 37 - protein, 43 aa, synthetic construct
  REGION 1..43: note = Synthetic sequence: Plant EOD domain
  VARIANT 1: note = X is Glu or Lys
  VARIANT 8: note = X is Leu or Met
  VARIANT 9: note = X is Lys, Arg, Gly, Thr or Glu
  VARIANT 11: note = X is Lys or Arg
  VARIANT 12: note = X is Arg or Ile
  VARIANT 13: note = X is Gly or Lys
  VARIANT 14: note = X is Asp, Asn or Glu
  VARIANT 15: note = X is Arg, Gln, Lys or Leu
  VARIANT 17: note = X is Ile, Met or Val
  VARIANT 18: note = X is Lys, Asn, Thr or Ala
  VARIANT 20: note = X is Leu or Pro
  VARIANT 22: note = X is Lys or Ser
  VARIANT 24: note = X is Val or Ala
  VARIANT 27: note = X is Ser, Thr, Gly or Ala
  VARIANT 28: note = X is Glu, Gln, Asp, Ser or Gly
  VARIANT 30: note = X is Ile, Gly, Thr or Val
  VARIANT 31: note = X is Ser or Thr
  VARIANT 32: note = X is Lys or Arg
  VARIANT 35: note = X is Gly, Thr or Ser
  VARIANT 39: note = X is Val, Ile, Ala or Lys
  VARIANT 42: note = X is Val or Ile
XRCVICQXXYXXXXXQXXLXCXHXYHXXCXXXWLXINKXCPXC

SEQ ID NO: 38 - protein, 203 aa, Populus trichocarpa
MEVHYMNTDFPYTTTESFMDFFEGLTHAPVNYAHNGPMHDQDNAYWSMNMNAYKFGFSGLGSTSYYSPYEVNDNLPRMDVSRMAWEYPSVVIKALWQDDVDPDTMTYEELVDLGETVGTQSKGLSPELISLLPTSKCKFGSFFSRKRSGERCVICQMKYKRGDKQIKLLCKHAYHSECITKWLGINKVCPVCNDEVFGEESRN

SEQ ID NO: 39 - protein, 240 aa, Ricinus communis
MEVHYINTGFPYTVTESFLDFFEGLSHVPVHYAHTGQVLDQVQENAYWSMNMNAYKYGFSGPGSTYYDPYEVNDNLPRMDVSRSTWEYPSVVNMEEATTTDTQSEGDAVVGVHASPEECIPNHTSGDSPQGVWQDDVDPDNMTYEELLDLGETVGTQSRGLSQELISLLPTSKCKFRSFFLRKKAGERCVICQMRYKRGDKQMKLPCKHVYHSECISKWLGINKVCPVCNNEVFGEDSRH

SEQ ID NO: 40 - protein, 245 aa, Prunus persica
MNGNGQMDVHYIDTDFPYTPTESFMDFFGGVTHVPMNYGHAMPMHDQETAYWSMNMHSYKFGPSGPGSNSYYGNYYEVNDHLPRMDVSRRTWEHPSVMNSEEPANIDSHPEEEDAVAEAAPEECIQNQQNTNTSQVVWQEDIDPDNMTYEELLDLGEAVGTQSRGLSDELISLLPTSKYKCGSFFSRKKSGERCVICQMRYKRGDRQINLPCKHVYHSECISKWLGINKVCPVCNLEVSGEESRH

SEQ ID NO: 41 - protein, 242 aa, Theobroma cacao
MNGNRQMEVHYIDTGFPYTATESFMDFFEGLTHVPVNYTHTVPMQDQENIYWSMSMNAYKFGFSGPESTFYSPYEVSDHLPRMDVSRRTWDYPSTLNSEEPATIDMQPGGEAVVGIHAIPEECITNHQSNSNSQVVWQDNIDPDNMTYEELLDLGETIGSQSRGLSQELIDLLPTSKCKFGSFFSTKRERCVICQMRYKRGEQQMKLPCKHVYHSQCITKWLSINKICPVCNNEVFGEESRH

SEQ ID NO: 42 - protein, 247 aa, Vitis vinifera
MNGNRQMEVHYINTGFPYTITESFMDFFEGLGHVPVNYAQAEAMHNQSIQENFYWTMNMNSYKFGFSGPGSTYYGPYDVNEHVPGIEVSRRPWEYPSSMIVEEPTTIETQPTGNEVMNVHAIPEECSPNHYSATSSQAIWQDNVDPDNMTYEELLDLGEAVGTQSRGLSQEHINLLPTCRYKSGRLFSRKRSAERCVICQMGYKRGDRQIKLPCKHVYHTDCGTKWLTINKVCPVCNIEVFGEESRH

SEQ ID NO: 43 - protein, 247 aa, Glycine max
MNDGRQMGVHYVDAGFPYAVNDNFVDFFQGFTHVPVNYAFAGSIPDQESVYWSMNMNPYKFGLSGPGSTSYYSSYEVNGHLPRMEIDRAEWEYPSTITTVEEPATTDSPPRRDGVTSMQTIPEECSPNHHESNSSSQVIWQDNIYPDDMTYEELLDLGEAVGTQSRGLSQELIDMLPTSKYKFGSLFKRKNSGKRCVICQMTYRRGDQQMKLPCSHVYHGECITKWLSINKKCPVCNTEVFGEESTH

SEQ ID NO: 44 - protein, 247 aa, Glycine max
MNDGRQMGVNYVDAGFPYAVNENFVDFFQGFTPVPVNYAFAGSIPDQESVYWSMNMNPYKFGLSGPGSTSYYSSYEVNGHLPRMEIDRAEWEYPSTITTVEEPATTDSPPRRDGVTNMQTIPEECSPNHHESNSSSQVIWQDNIDPDNMTYEELLDLGEAVGTQSRGLSQELIDMLPTSKYKFGNLFKRKNSGKRCVICQMTYRRGDQQMKLPCSHVYHGECITKWLSINKKCPVCNTEVFGEESTH

SEQ ID NO: 45 - protein, 248 aa, Arabidopsis thaliana
MNGDNRPVEDAHYTETGFPYAATGSYMDFYGGAAQGPLNYDHAATMHPQDNLYWTMNTNAYKFGFSGSDNASFYGSYDMNDHLSRMSIGRTNWDYHPMVNVADDPENTVARSVQIGDTDEHSEAEECIANEHDPDSPQVSWQDDIDPDTMTYEELVELGEAVGTESRGLSQELIETLPTKKYKFGSIFSRKRAGERCVICQLKYKIGERQMNLPCKHVYHSECISKWLSINKVCPVCNSEVFGEPSIH

SEQ ID NO: 46 - protein, 243 aa, Capsella rubella
MNGDRPVEDAHYTEAEFPYAASGSYIDFYGGAPQGPLNYAHAGTMDNLYWTMNTNAYKFGFSGSDNPSFYNSYDMTDHLSRMSIGRTNWEYHPMVNVDDPDITLARSVQIGDSDEHSEAEDCIANEHDPDSPQVSWQDDIDPDTMTYEELVELGEAVGTESRGLSQELIETLPTRKFKFGSIFSRKRAGERCVICQLKYKIGERQMNLPCKHVYHSECISKWLSINKVCPVCNTEVFGDPSIH

SEQ ID NO: 47 - protein, 249 aa, Sorghum bicolor
MNSCRQMELHYINTGFPYTITESFMDFFEGLTYAHADFALMDGFQDQGNPYWAMMHTNSYKYGYSGPGNYYTYAHVYDIDDYMHRADGGRRVWDNTTPANNVDSANVVLQGSEAPRTTANTTTEECIQQVHQSPGSPHVVWQDNIDPDNMTYEELLDLGEVVGTQSRGLSQERISSLPVTKYKCGFFSRKKTRRERCVICQMEYRRGNLQMTLPCKHVYHASCVTRWLSINKVCPVCFAEVPGDEPKRQ

SEQ ID NO: 48 - protein, 253 aa, Zea mays
MNSSRQMELHYINTGFPYTITESFMDFFEGLTYAHADFALTDGFQDQGNPYWAMMHTNSYKYGYSGPGNYYSYAHVYDIDDYMRRADGGRRIWDNTTPVNNVDSANVVLQGGEAPHTTTNTINKECIQQVHQSPGSPQVVWQDNIEPDNMTYEELLDLGEAVGTQSRGLSQERISSLPVTKYKCGFFSRKKTRRERCVICQMEYRRGNLQMTLPCKHVYHASCVTRWLGINKVCPVCFAEVPGEDPEAMSQQL

SEQ ID NO: 49 - protein, 249 aa, Zea mays
MTSSRQMELHYINTGFPYTITESFMDFFEGLTYAHADFALMDGFQDQGNPYWTMMHTNSYKYGYSGSGNYYSYAHAYDIDDYMHRTDGGRRTWDNTTPVNNVDSANVVLQGGEAPRTTANTTSEDCIQQVHQSPGSPQVVWQDNIDPDNMTYEELLDLGEAVGTQSRGLSQECISLLPITKYKCGFFSRKKTRRERCVICQMEYRRGNLQITLPCKHVYHASCVTRWLSINKVCPVCFAEVPGEDSLRQ

SEQ ID NO: 50 - protein, 272 aa, Oryza sativa
MTESHERDTEVTRWQVHDPSEGMNGSRQMELHYINTGFPYTITESFMDFFEGLTYAHADFAIADAFHDQANPYWAMMHTNSYKYGYSGAGNYYSYGHVYDMNDYMHRADGGRRIWDNATPVNNTESPNVVLQGGETPHANTSSTTEECIQQQVHQNSSSPQVIWQDNIDPDNMTYEELLDLGEAVGTQSRGLSQERISLLPVTKYKCGFFSRKKTRRERCVICQMEYRRGNLQMTLPCKHVYHASCVTRWLSINKVCPVCFAEVPGDEPKRQ

SEQ ID NO: 51 - protein, 250 aa, Oryza sativa
MNGSRQMELHYINTGFPYTITESFMDFFEGLTYAHADFAIADAFHDQANPYWAMMHTNSYKYGYSGAGNYYSYGHVYDMNDYMHRADGGRRIWDNATPVNNTESPNVVLQGGETPHANTSSTTEECIQQQVHQNSSSPQVIWQDNIDPDNMTYEELLDLGEAVGTQSRGLSQERISLLPVTKYKCGFFSRKKTRRERCVICQMEYRRGNLQMTLPCKHVYHASCVTRWLSINKVCPVCFAEVPGDEPKRQ

SEQ ID NO: 52 - protein, 251 aa, Brachypodium distachyon
MNGSRQMELHYINTGFPYTITESFMDFFEGLTYAHADFALADAFQDQANPYWTMMQTNSYKYGYSGASNYYSYGHVYDMNDYMHRADGGRRIWDNPTPASNTDSPNVVLQGAAEAPHPRASSTTEECIQQPVHQNSSSPQVVWQDNVDPDNMTYEELLDLGEAVGTQSRGLSQERISSLPVTKYKCGFFSRKKTRRERCVICQMEYRRGDLQMALPCKHVYHASCVTRWLSINKVCPVCFAEVPSEEPSRQ

SEQ ID NO: 53 - protein, 246 aa, Solanum lycopersicum
MNWNQQTEIYYTNGAMPYNSIGSFMDFFGGVTYDHVNYIFADPPYAQESLYPSISTNPYKFGYSEAGSFSYYDYDREYVVNDHVSGIEEHDRHLENPSTTTVNVAANVHREEISGSNSLTNSVECPRGQINTRDSEVVWQDNIDPDNMTYEELLELGEAVGTQSRGLSQNQISLLPVTKFKCGFFSRKKSRKERCVICQMEYKRKDQQVTLPCKHVYHAGCGSRWLSINKACPICYTEVVINTSKR

SEQ ID NO: 54 - protein, 10 aa, synthetic construct
  REGION 1..10: note = Synthetic sequence: LIM domain motif
HXXCXXCXXC